(19) United States
(12) Patent Application Publication
(10) Pub. No.: US 2023/0398040 A1
(43) Pub. Date: Dec. 14, 2023

(54) A MULTI-LAYERED SHEET MASK

(71) Applicant: NATIONAL UNIVERSITY OF SINGAPORE, Singapore (SG)

(72) Inventors: Yin Chiang Freddy BOEY, Singapore (SG); Ngeow Khing CHIA, Singapore (SG); Tien Guan THNG, Singapore (SG)

(73) Assignee: NATIONAL UNIVERSITY OF SINGAPORE, Singapore (SG)

(21) Appl. No.: 18/036,128

(22) PCT Filed: Nov. 9, 2021
(86) PCT No.: PCT/SG2021/050685

§ 371 Date: May 9, 2023

Related U.S. Application Data
(60) Provisional application No. 63/198,726, filed on Nov. 9, 2020.

(51) Int. Cl. A61K 8/02; A61M 35/00; A61Q 19/00; A61K 8/66; A61K 8/73; A45D 44/00

(52) U.S. Cl. CPC A61K 8/0212; A61M 35/10; A61Q 19/00; A61K 8/66; A61K 8/735; A45D 44/002; A61M 2207/10; A61M 2210/0606

(57) ABSTRACT

There is provided a multi-layered sheet mask comprising at least (a) a porous first layer having a first side and a second side, wherein the first side is opposite to the second side, and comprises a plurality of three-dimensional patterns extending therefrom; (b) a second layer attached to the second side of the porous first layer, the second layer comprising at least one pouch for receiving or containing at least one first active ingredient; a porous support structure; an air pouch that is sandwiched between the second side of the porous first layer and the porous support structure; and a plurality of microchannels that extend from the pouch and beyond the first side of the porous first layer when the air pouch is deflated; and (c) a third layer attached to the second layer for receiving or containing at least one second active ingredient. There is also provided a mould for producing the three-dimensional patterns of the porous first layer of the multi-layered sheet mask, and a method of producing the multi-layered sheet mask.

### REFERENCES TO RELATED APPLICATION

This application claims priority to U.S. provisional application No. 63/198,726 filed on 9 Nov. 2020, the disclosure of which is hereby incorporated by reference.

### FIELD OF THE INVENTION

The present invention relates generally to the field of a multi-layered sheet mask for provision of multiple cosmetic functions such as skin exfoliation, hydration, brightening, rejuvenation, treatment, anti-acne, anti-ageing, and anti-wrinkle. In particular, the present invention relates to a multi-layered sheet mask, a method of producing the multi-layered sheet mask, and a mould for producing the three-dimensional patterns of the porous first layer of the multi-layered sheet mask.

### BACKGROUND ART

The human face is exposed to indoor and outdoor environments daily and directly. Many studies have been done to investigate how environmental factors such as temperature, humidity, sun exposure, and air pollutions levels could affect our facial skin. It has been observed that during warm weather, the facial skin tends to be oilier, and both dry and cold weathers could cause skin dryness and irritation. Unprotected facial skin tends to age and darken faster when subjected to long-term sun exposure. Therefore, to maintain the health of facial skin and to reduce the above-mentioned negative effects of skin exposure to a non-ideal environment, skincare is needed to prevent or mitigate such negative effects.

Among many facial skincare products, a sheet mask is one that comes commonly packaged for single use and is widely considered more hygienic, as compared to a reusable face mask that is packaged in a pot or tube. A sheet mask impregnated with cosmetic compositions can be used for moisturizing, oil removing, and improving the overall aesthetics of the face whilst providing user comfort. Although a sheet mask can provide multiple functions, the most important function is to ensure that the cosmetic compositions can be absorbed by the skin layers, especially the subcutaneous layers of the skin.

It is known that skin has three layers: the deeper subcutaneous tissue, the dermis, and the epidermis. The deeper subcutaneous tissue is made of fat and connective tissue. The dermis, atop the deeper subcutaneous tissue, contains tough connective tissues, hair follicles, and sweat glands. The epidermis is the outermost layer of the skin, providing a waterproof barrier and forms the skin tone.

Skin absorption is a route by which cosmetic compositions that are beneficial for facial skin can enter into the deeper layer of skin, and further into the body systemic circulation from the outer surface of the skin Absorption of cosmetic compositions through the skin is affected by a number of factors, such as concentration, duration of contact, solubility, physical condition of the skin, and the specific part of the body where the skin is located. However, one of the most important factors limiting skin absorption rate relates to the stratum corneum, which is the outermost layer of the epidermis layer of skin that acts as a barrier to protect underlying tissue from infection, dehydration, foreign substances, and mechanical stress. Therefore, how quickly cosmetic compositions can penetrate the stratum corneum determines the efficiency of skin absorption.

The stratum corneum is primarily composed of lipophilic cholesterol, cholesterol esters, and ceramides and generally has a thickness between 10 μm and 40 μm, comprising 15 to 20 layers of dead skin cells. Thus, lipid-soluble cosmetic composition can pass through the stratum corneum faster; however, as lipophilic molecules can only passively diffuse through the layers, the resultant lipid-soluble cosmetic composition that penetrates skin is mere to some minimal degrees.

In consideration of the above, exfoliation is necessary to improve the efficiency of skin absorption. Exfoliation is a process of removing dead skin cells from the epidermal layer of facial skin. Conventionally, exfoliation can be done in two ways: mechanical exfoliation and chemical exfoliation.

In mechanical exfoliation, a tool (e.g., a brush or a washcloth) or facial scrub physically removes the dead skin cells. Mechanical beads or exfoliating particles are mixed into a facial scrub cream or embedded in a membrane. This process involves physically scrubbing the skin with an abrasive. Mechanical exfoliators include microfiber cloths, adhesive exfoliation sheets, micro-bead facial scrubs, crepe paper, crushed apricot kernel or almond shells, sugar, or natural minerals like salt crystals, pumice, and abrasive materials such as sponges, loofahs, and brush-like needles. Facial scrubs are commonly available in over-the-counter products. However, persons with dry skin should avoid mechanical exfoliators which include a significant portion of pumice or crushed volcanic rock.

On the other hand, for chemical exfoliation, the chemical exfoliant comprises ingredients such as alpha- or beta-hydroxy acids (e.g., a facial wash with salicylic acid, or a peel pad with glycolic acid) to remove dead skin cells. Chemical exfoliants include facial wash or scrub containing salicylic acid, glycolic acid, fruit enzymes, citric acid, or malic acid which may be applied in high concentrations by a medical professional, or in lower concentrations in over-the-counter products. Chemical exfoliation that involves the use of products that contain alpha- or beta-hydroxy acids or enzymes acts to loosen a glue-like substance that holds the dead skin cells together, allowing the dead skin cells to be dislodged. This type of exfoliation is recommended for persons with acne condition or physical defects on the facial skin.

While there are cosmetic sheet masks with exfoliation function available on the market, they are made of sheets of even thickness. However, the thickness of the human facial skin is not homogenous. According to research conducted to examine the topographic thickness of facial skin, results show that the thickness of facial skin and the thickness of the epidermis layer of facial skin are not the same on one face. The thickest human facial skin is found at the lower third of the nose (specifically the lower nasal sidewall), and the thinnest facial skin is found at the medial aspect of the upper eyelid, while the thickest epidermis is found at the upper lip, and the thinnest epidermis is found at the posterior auricular region. Hence, a deeper exfoliation is needed at a thicker skin area of the face, so that the same quantity of cosmetic composition can be effectively absorbed as compared to a thinner skin area of the face. This is to ensure that a homogenous cosmetic effect can be effectively applied to the different areas of facial skin which is of varying skin thickness.

Further, most cosmetic sheet masks on the market have a single main function. Hence, to achieve multiple cosmetic functions on a facial skin, for instance exfoliation, hydration and ultraviolet-light protection, it would normally be required to take multiple separate steps, for example exfoliation followed by hydration followed by application of an ultraviolet-light protection serum, which may require multiple sheet masks and/or combination with other forms of cosmetic products.

Thus, there is a need to develop a multi-layered sheet mask that overcomes or at least ameliorates, one or more limitations with existing technologies. The multi-layered sheet mask is preferably one that has multiple functions and simultaneously targets skin surface composed of varying skin thickness. There is also a need to provide a method for preparing such a multi-layered sheet mask.

### SUMMARY

In one aspect, the present disclosure refers to a multi-layered sheet mask comprising at least
a) a porous first layer having a first side and a second side, wherein the first side is opposite to the second side, and comprises a plurality of three-dimensional patterns extending therefrom;
b) a second layer attached to the second side of the porous first layer, the second layer comprising at least one pouch for receiving or containing at least one first active ingredient; a porous support structure; an air pouch that is sandwiched between the second side of the porous first layer and the porous support structure; and a plurality of microchannels that extend from the pouch and beyond the first side of the porous first layer when the air pouch is deflated; and
c) a third layer attached to the second layer for receiving or containing at least one second active ingredient.

Advantageously, each layer of the multi-layered sheet mask may provide at least one cosmetic function to the facial skin. For example, the porous first layer of the multi-layered sheet mask may exfoliate the facial skin, causing the face to be brightened as the dead skin cells are removed. However, as exfoliation may cause minor forms of redness to appear on the facial skin, moisturization of the exfoliated skin is required which may be provided by the first active ingredient in the second layer of the multi-layered sheet mask. Further, the second layer of the multi-layered sheet mask may concurrently deliver active ingredients such as collagen via the microchannels to the dermis layer of the facial skin to stimulate collagen growth. After the facial skin has fully absorbed a moisturizing agent and a collagen growth stimulant, for example, an oil-based cosmetic such as facial oil as the second active ingredient, may be dispensed through the third layer of the multi-layered sheet mask to the facial skin. The active ingredients provided by the second and third layers of the sheet mask may be tailored according to the specific needs of the users.

Further advantageously, the multi-layered sheet mask may provide multiple cosmetic functions in a single mask, for instance, exfoliation and other cosmetic enhancement purposes may be achieved in one mask, as opposed to having to use several different single-layered masks. Hence, the multi-layered sheet mask offers more convenience and time efficiency as compared to use of several different single-layered masks.

Further advantageously, the multi-layered sheet mask may ensure higher penetration of active ingredients to the dermis layer of the facial skin due to the combined effects of exfoliation, where dead skin cells are removed for better absorption of active ingredients, and the delivery of active ingredients through the microchannels that penetrates the dermis layer of the facial skin.

Further advantageously, the multi-layered sheet mask may allow specific targeting of different zones of the facial skin by skin thickness or skin condition, thus allowing the multi-layered sheet mask to achieve enhanced cosmetic effects on specific users.

In another aspect, the present disclosure refers to a mould for producing the three-dimensional patterns of the porous first layer of the multi-layered sheet mask as disclosed herein, wherein the mould comprises a negative replica of the three-dimensional patterns.

Advantageously, the mould may be designed to comprise a negative replica of the three-dimensional patters of the porous first layer of the multi-layered sheet mask, which is dedicated for specific zones of the facial skin based on skin thickness and/or skin condition.

Further advantageously, the mould may allow the facile reproduction of complex micrometre-scale three-dimensional structures as exfoliators for cosmetic purposes.

In another aspect, the present disclosure refers to a method of producing a multi-layered sheet mask, the method comprising the steps of:
a) forming a porous first layer having a first side and a second side, by contacting a mask-shaped biocompatible material with a whole facial mask mould having a plurality of three-dimensional patterns thereon, wherein the three-dimensional patterns comprise a material selected from the group comprising of biocompatible material, exfoliating agent and combinations thereof, and drying for a duration;
b) attaching the second layer to the second side of the porous first layer by bonding the circumferential edges of the first and second layers together; and
c) attaching a third layer to the second layer by bonding the circumferential edges of the second and third layers together;
   wherein the multi-layered sheet mask comprises at least
a porous first layer wherein the first side is opposite to the second side and comprises a plurality of three-dimensional patterns extending therefrom;
a second layer attached to the second side of the porous first layer, the second layer comprising at least one pouch for receiving or containing at least one first active ingredient; a porous support structure; an air pouch that is sandwiched between the second side of the porous first layer and the porous support structure; and a plurality of microchannels that extend from the pouch and beyond the first side of the porous first layer when the air pouch is deflated; and
a third layer attached to the second layer for receiving or containing at least one second active ingredient.

Further advantageously, the method may be adaptable to create additional layers, for example the fourth or fifth layers of mask, to offer additional cosmetic treatment for the facial skin. The method may also be adaptable to create multi-layered sheet mask for other parts of the body besides the face.

### DEFINITIONS

The following words and terms used herein shall have the meaning indicated:

The term “microchannel” as used herein refers to a channel with dimensions, such as length, diameter, width and height, in the micrometre scale, with a pointed tip on one end.

The term “pouch” as used herein refers to a pocket or bag for containing a liquid or gaseous substance.

The term “positive replica” as used herein refers to an exact structural copy of identical shape and contours. When a positive replica is used as a mould to create a structural product, the structural product will thus bear the shape and contours of a negative replica.

The term “negative replica” as used herein refers to an exact reverse structural copy of shape and contours. When a negative replica is used as a mould to create a structural product, the structural product will thus bear the shape and contours of a positive replica.

The term “UV-curable” as used herein refers to a material that can be treated by ultraviolet radiation to generate a crosslinked network of polymers, thus hardening or toughening in the process.

The term “prism” as used herein refers to a three-dimensional shape with two identical polygon bases facing each other, such that each polygon base can be a regular or irregular polygon.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means+/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### DETAILED DISCLOSURE OF EMBODIMENTS

Exemplary, non-limiting embodiments of a multi-layered sheet mask will now be disclosed.

The multi-layered sheet mask comprises at least
a) a porous first layer having a first side and a second side, wherein the first side is opposite to the second side, and comprises a plurality of three-dimensional patterns extending therefrom;
b) a second layer attached to the second side of the porous first layer, the second layer comprising at least one pouch for receiving or containing at least one first active ingredient; a porous support structure; an air pouch that is sandwiched between the second side of the porous first layer and the porous support structure; and a plurality of microchannels that extend from the pouch and beyond the first side of the porous first layer when the air pouch is deflated; and
c) a third layer attached to the second layer for receiving or containing at least one second active ingredient.

The porous first layer and the porous support structure of the second layer may comprise a plurality of slits.

Advantageously, each layer of the multi-layered sheet mask may provide at least one cosmetic function to the facial skin. For example, the porous first layer of the multi-layered sheet mask may exfoliate the facial skin, causing the face to be brightened as the dead skin cells are removed. However, as exfoliation may cause minor forms of redness to appear on the facial skin, moisturization of the exfoliated skin is required which may be provided by the first active ingredient in the second layer of the multi-layered sheet mask. Further, the second layer of the multi-layered sheet mask may concurrently deliver active ingredient such as collagen via the microchannels to the dermis layer of the facial skin to stimulate collagen growth. After the facial skin has fully absorbed a moisturizing agent and a collagen growth stimulant, for example, an oil-based cosmetic such as facial oil as the second active ingredient, may be dispensed through the third layer of the multi-layered sheet mask to the facial skin. The active ingredients provided by the second and third layers of the sheet mask may be tailored according to the specific needs of the users.

Further advantageously, the multi-layered sheet mask may provide multiple cosmetic functions in a single mask, for instance, exfoliation and other cosmetic enhancement purposes may be achieved in one mask, as opposed to having to use several different single-layered masks. Hence, the multi-layered sheet mask offers more convenience and time efficiency as compared to use of several different single-layered masks.

Further advantageously, the multi-layered sheet mask may ensure higher penetration of active ingredients to the dermis layer of the facial skin due to the combined effects of exfoliation, where dead skin cells are removed for better absorption of active ingredients, and the delivery of active ingredients through the microchannels that penetrates the dermis layer of the facial skin.

Further advantageously, the multi-layered sheet mask may allow specific targeting of different zones of the facial skin by skin thickness or skin condition, thus allowing the multi-layered sheet mask to achieve enhanced cosmetic effects on specific users.

The multi-layered sheet mask may be a facial mask providing cosmetic care to a recipient of the multi-layered sheet mask. The multi-layered sheet mask may also be used as a mask on other parts of a recipient's body. The multi-layered sheet mask may be a hand mask, a foot mask or a body mask.

The multi-layered sheet mask may be designed to have a boundary edge so that during application of the mask, the active ingredients may not affect the natural orifice of the recipient or flow to contaminate areas of the recipient's body that do not need the active ingredients.

The porous first layer of the multi-layered sheet mask may have a first side and a second side, the first side contacting a facial skin (or a body part of a recipient).

The three-dimensional patterns may have an end integrated to the first side of the porous first layer of the multi-layered sheet mask or positioned within the porous first layer and the other end protruding from the first side toward the facial skin (or a body part of a recipient) when the air pouch is deflated.

The porous first layer and the three-dimensional patterns may be made of a material independently selected from the group consisting of synthetic, regenerated and natural biocompatible materials. The porous first layer may be made of a material which may be the same or different from the three-dimensional patterns.

The material may be selected from the group consisting of UV-curable polymer, UV-LED curable polymer, bioabsorbable polymer, cotton, nylon, nylon microfibre, regenerated cellulose fibre, cellulose, biocellulose, foil, hyaluronic acid, and hydrogel. The regenerated cellulose fibre may be Cupro, Tencel, Modal, Lyocell, Viscose or Rayon.

The UV-curable polymer may be polyvinyl alcohol.

The foil may be a gold foil. Masks that contain gold foil are able to trap heat to open up skin pores for superior delivery of the active ingredient(s). Normally, the foil is outside away from the facial skin (or a body part of a recipient) and the first layer (which can be soft cellulose or a similar material) is in contact with the facial skin (or a body part of a recipient) to deliver the active ingredient(s). The three-dimensional patterns can be cast on the inside of the first layer in contact with the facial skin (or a body part of a recipient).

When the material used is hyaluronic acid, the molecular weight of the hyaluronic acid may be varied to control the time for its dissolution. Hyaluronic acid (HA) includes hyaluronan or hyaluronate and its derivates and their application in cosmetic formulations. HA is a glycosaminoglycan constituted from two disaccharides (N-acetylglucosamine and D-glucuronic acid). HA may be crosslinked by using a photoinitiator such as lithium phenyl(2,4,6-trimethylbenzoyl)phosphinate (LAP). Another example is Hyaluronic Acid-aldehyde crosslinked with O,O′-1,3 propanediylbishydroxylamine dihydrochloride (POA) in the presence of standard HA. HA can have a wide range of molecular weights ranging from 20,000 to 10,000,000 Da. HA may be used to form the three-dimensional patterns on the first layer whereby the three-dimensional patterns are made completely of HA with different layer of HA with different molecular weights. In one embodiment, the HA can be cast on a cellulose first layer which is porous. In use, when such a mask is pressed against the surface of a face, the HA can provide the exfoliation and open up the surface pores while the active ingredient(s) from the second layer elutes through the first layer, the HA dissolves within the skin and thereby becomes absorbed by the skin. In a similar manner, any material that is able to dissolve can be used to form the three-dimensional patterns, or a mixture of dissolving materials can be used to form the three-dimensional patterns.

The hydrogel may be woven or non-woven. The hydrogel may be gelatin methacryloyl (GelMA) hydrogel. The GelMA hydrogel may be mixed with a suitable photoinitiator such as Irgacure 2959 or lithium phenyl-2,4,6-trimethylbenzoylphosphinate (LAP) prior to light-induced polymerization.

Stratum corneum, as the outermost layer of the epidermis, is a rate-limiting barrier for facial skin absorption. The three-dimensional patterns may be used to provide mechanical exfoliation, whereby the physical structure and shape of the three-dimensional patterns influence the mechanical exfoliation effect. The three-dimensional pattern may comprise an exfoliating agent to provide chemical exfoliation. Both mechanical exfoliation and chemical exfoliation may be provided simultaneously by the three-dimensional patterns to facial skin. Thus, the three-dimensional patterns may physically remove dead skin cells in the stratum corneum by mechanical and/or chemical exfoliation, thus increasing the rate of facial skin absorption of active ingredient(s). Advantageously, the exfoliation action of the three-dimensional patterns may improve the appearance of uneven skin tone and bring radiance to a dull complexion.

The three-dimensional patterns may comprise an exfoliating agent selected from the group consisting of alpha hydroxy acids, beta hydroxy acids, plant-based enzymes, animal-based enzymes and mixtures thereof; or wherein the three-dimensional patterns may comprise an exfoliating agent selected from the group consisting of lactic acid, last obionic acid, glycolic acid, hydroxycaproic acid, hydroxycaprylic acid, citric acid, malic acid, mandelic acid, tartaric acid, phytic acid, salicylic acid, hyaluronic acid, azelaic acid, kojic acid, ascorbic acid, trichloroacetic acid, alguronic acid, lipoic acid, ferulic acid and mixtures thereof.

Lactic acid may provide many of the same benefits as other alpha hydroxyl acids but may be safe for use on sensitive skin since lactic acid works by breaking down and dissolving dead skin cells without causing irritation through a long period of application, depending on the molecular weight of the lactic acid. The lactic acid to be incorporated in the porous first layer of the multi-layered sheet mask may be obtained by degradation of poly(lactic acid) of molecular weight 5000 g/mol into degradation products of less than about 100 g/mol. The degradation of poly(lactic acid) may be via a biodegradation process.

Further, lactic acid to be incorporated in the porous first layer may prevent facial skin from dehydration and keep the skin moisturized. As molecules of different molecular weights may penetrate to different depths of the skin, molecules with a lower molecular weight may reach deeper layer(s) of skin, thus sustaining the moisture content of the skin at deeper layer(s). Further, lactic acid to be incorporated in the porous first layer may reduce the signs of ageing by improving the appearance of fine lines. In addition, lactic acid may increase facial skin firmness and thickness. Thus, the primary benefits of lactic acid for skin may include: 1) brightening and evening skin tone, 2) stimulating skin cell turnover and renewal; 3) revealing glowing facial surfaces and 4) anti-ageing effect to result in youthful looking skin.

For hyaluronic acid, the average molecular weight may be in a range of about 1,000,000 g/mol to about 8,000,000 g/mol, about 1,000,000 g/mol to about 6,000,000 g/mol, about 1,000,000 g/mol to about 5,000,000 g/mol, about 1,000,000 g/mol to about 4,000,000 g/mol, about 1,000,000 g/mol to about 2,000,000 g/mol, about 2,000,000 g/mol to about 8,000,000 g/mol, about 2,000,000 g/mol to about 6,000,000 g/mol, about 2,000,000 g/mol to about 5,000,000 g/mol, about 2,000,000 g/mol to about 4,000,000 g/mol, about 4,000,000 g/mol to about 8,000,000 g/mol, about 4,000,000 g/mol to about 6,000,000 g/mol, about 4,000,000 g/mol to about 5,000,000 g/mol, about 5,000,000 g/mol to about 8,000,000 g/mol, about 5,000,000 g/mol to about 6,000,000 g/mol, or about 6,000,000 g/mol to about 8,000,000 g/mol.

The three-dimensional patterns may have an average diameter in the range of about 1 μm to about 1 mm, about 10 μm to about 1 mm, about 50 μm to about 1 mm, about 100 μm to about 1 mm, about 500 μm to about 1 mm, about 1 μm to about 10 μm, about 1 μm to about 50 μm, about 1 μm to about 100 μm, about 1 μm to about 500 μm, about 10 μm to about 50 μm, about 10 μm to about 100 μm, about 10 μm to about 500 μm, about 50 μm to about 100 μm, about 50 μm to about 500 μm, or about 100 μm to about 500 μm.

The three-dimensional patterns may have a height in the range of about 1 mm to about 3 mm, about 1.2 mm to about 3 mm, about 1.5 mm to about 3 mm, about 2 mm to about 3 mm, about 2.5 mm to about 3 mm, about 1 mm to about 1.2 mm, about 1 mm to about 1.5 mm, about 1 mm to about 2 mm, about 1 mm to about 2.5 mm, about 1.2 mm to about 1.5 mm, about 1.2 mm to about 2 mm, about 1.2 mm to about 2.5 mm, about 1.5 mm to about 2 mm, about 1.5 mm to about 2.5 mm, or about 2 mm to about 2.5 mm.

In general, the stratum corneum has a thickness between 10 μm and 40 μm. For the three-dimensional patterns that only conduct mechanical exfoliation, the height of the three-dimensional patterns may be designed to penetrate the stratum corneum but not penetrate to the dermis layer of the facial skin. Therefore, the dead skin cells in stratum corneum may be removed by the three-dimensional patterns without hurting the dermis layer.

The three-dimensional patterns may have a height designed based on a typical topographic thickness of the stratum corneum of the facial skin to achieve a homogenous penetration depth in different zones of the facial skin to maximize exfoliation. Thus, the height of the three-dimensional patterns may be different for different zones of the facial skin.

The three-dimensional patterns may comprise a plurality of pointed three-dimensional shapes as skin-engaging ends, wherein the pointed three-dimensional shapes may be selected from the group consisting of cone, pyramid, stellated polyhedron, regular polyhedron, irregular polyhedron partial regular polyhedron, partial irregular polyhedron, sphere and at least one cone, sphere and at least one spike, sphere and at least one pyramid, hemisphere and at least one spike, spike, and combinations thereof.

The pointed three-dimensional shapes may comprise the exfoliating agent.

The pointed three-dimensional shapes may have a height in the range of about 200 μm to about 250 μm, about 210 μm to about 250 μm, about 220 μm to about 250 μm, about 230 μm to about 250 μm, about 240 μm to about 250 μm, about 200 μm to about 210 μm, about 200 μm to about 220 μm, about 200 μm to about 230 μm, about 200 μm to about 240 μm, about 210 μm to about 220 μm, about 210 μm to about 230 μm, about 210 μm to about 240 μm, about 220 μm to about 230 μm, about 220 μm to about 240 μm, or about 230 μm to about 240 μm.

When the pointed three-dimensional shapes comprise of at least one spike, each spike may have a length in the range of about 40 μm to about 100 μm, about 50 μm to about 100 μm, about 60 μm to about 100 μm, about 80 μm to about 100 μm, about 40 μm to about 50 μm, about 40 μm to about 60 μm, about 40 μm to about 80 μm, about 50 μm to about 60 μm, about 50 μm to about 80 μm, or about 60 μm to about 80 μm.

The spike may be further positioned at an angle in the range of about 10° to about 30°, about 15° to about 30°, about 20° to about 30°, about 25° to about 30°, about 10° to about 15°, about 10° to about 20°, about 10° to about 25°, about 15° to about 20°, about 15° to about 25°, or about 20° to about 25° from the vertical plane.

When the spike is positioned at an angle from the vertical plane, the spike may have higher penetration area from the angle and from an adjacent area of a center spike, as compared to a spike that is not positioned at an angle from the vertical plane.

Further, when the spike is positioned at an angle from the vertical plane, the spike may have a different depth of penetration when the first layer of the multi-layered sheet mask is compressed, as compared to a spike that is not positioned at an angle from the vertical plane.

The slits of the porous first layer may extend from the second side of the porous first layer to the first side of the porous first layer. The slits of the porous support structure of the second layer may extend from one side to another side of the porous support structure.

The slits of the porous first layer and slits of the porous support structure of the second layer may have an inner diameter in the range of about 1 μm to about 100 μm, about 10 μm to about 100 μm, about 20 μm to about 100 μm, about 50 μm to about 100 μm, about 80 μm to about 100 μm, about 10 μm to about 20 μm, about 10 μm to about 50 μm, about 10 μm to about 80 μm, about 20 μm to about 50 μm, about 20 μm to about 80 μm, or about 50 μm to about 80 μm.

The slits of the porous first layer and slits of the porous support structure of the second layer may have an outer diameter in the range of about 100 μm to about 150 μm, about 110 μm to about 150 μm, about 120 μm to about 150 μm, about 130 μm to about 150 μm, about 140 μm to about 150 μm, about 100 μm to about 110 μm, about 100 μm to about 120 μm, about 100 μm to about 130 μm, about 100 μm to about 140 μm, about 110 μm to about 120 μm, about 110 μm to about 130 μm, about 110 μm to about 140 μm, about 120 μm to about 130 μm, about 120 μm to about 140 μm, or about 130 μm to about 140 μm.

The slits of the porous first layer and slits of the porous support structure of the second layer may function as paths to allow the active ingredient(s) to flow to the facial skin. When the at least one pouch in the second layer is punctured by the plurality of microchannels when the air pouch is deflated, the slits of the porous support structure of the second layer may help direct the at least one first active ingredient from the at least one pouch to the porous first layer The at least one first active ingredient may be further directed by the slits of the porous first layer to the surface of the skin for skin absorption.

The slits of the porous first layer and slits of the porous support structure of the second layer may have different shapes to control the flow rate of active ingredient(s) passing through the slits. Thus, the active ingredient(s) may flow through the slits of the porous first layer and slits of the porous support structure of the second layer at a controlled flow rate and be delivered to the recipient of the multi-layered sheet mask.

The flow rate of the active ingredient(s) through the slits of the porous first layer and slits of the porous support structure of the second layer may be in a range of about 0.1 ml/min to about 100 ml/min, about 1 ml/min to about 100 ml/min, about 10 ml/min to about 100 ml/min, about 20 ml/min to about 100 ml/min, about 50 ml/min to about 100 ml/min, about ml/min to about 100 ml/min, about 0.1 ml/min to about 1 ml/min, about 0.1 ml/min to about 10 ml/min, about 0.1 ml/min to about 20 ml/min, about 0.1 ml/min to about 50 ml/min, about 0.1 ml/min to about 80 ml/min, about 1 ml/min to about 10 ml/min, about 1 ml/min to about 20 ml/min, about 1 ml/min to about 50 ml/min, about 1 ml/min to about 80 ml/min, about 10 ml/min to about 20 ml/min, about 10 ml/min to about 50 ml/min, about 10 ml/min to about 80 ml/min, about 20 ml/min to about 50 ml/min, about 20 ml/min to about 80 ml/min, or about 50 ml/min to about 80 ml/min.

The slits of the porous first layer and slits of the porous support structure of the second layer may have a shape selected from the group consisting of polyhedron, non-polyhedron, and combinations thereof; or wherein the slits of the porous first layer and slits of the porous support structure of the second layer may have a shape selected from the group consisting of cylinder, prism, and combinations thereof; or wherein the slits of the porous first layer and slits of the porous support structure of the second layer may have a hexagonal prism shape. Collectively, an area of the slits of the porous first layer and an area of the slits of the porous support structure of the second layer may be viewed as a honeycomb structure.

The microchannels may penetrate through the three-dimensional patterns if the three-dimensional patterns are designed to have a hollow center for microchannels to penetrate through. In this instance, the three-dimensional patterns may provide exfoliation and insert first active ingredient to the dermis layer of the facial skin, wherein the microchannels may have one end inserted in the pouch in the second layer and have the other end penetrate the dermis layer of the skin when the air pouch is deflated. Otherwise, the three-dimensional patterns without microchannels penetrating through are non-hollow three-dimensional patterns for conducting exfoliation, such that on the facial skin, the microchannel may be adjacent to the non-hollow three-dimensional patterns for near-site dispensing of first active ingredient.

The microchannels may comprise pointed tips which penetrate the second side of the porous first layer, the first side of the porous first layer, an epidermis layer of a skin, and a dermis layer of a skin, when the air pouch is deflated.

The microchannels may have a height designed based on a typical topographic thickness of the human facial skin to achieve a homogenous penetration depth in different zones of the facial skin, when the mask to used on a human. Thus, the height of the microchannels may be different for different zones of the facial skin. This may enable the first and/or second active ingredients to be delivered to a similar depth in different zones of the facial skin, allowing the skin cells to receive the active ingredient(s) evenly.

The microchannels of the second layer of the mask may have a height corresponding to different zones of a face as follows:
a height in the range of about 1.5 mm to about 1.8 mm corresponding to a first zone consisting of hairline, forehead, temple and combinations thereof of a face;
a height in the range of about 1.2 mm to about 1.5 mm corresponding to a second zone consisting of nose, cutaneous upper lip, philtum, philtum crest, cutaneous lower lip, chin, and combinations thereof of a face; and
a height in the range of about 1.6 mm to about 1.9 mm corresponding to a third zone consisting of cheeks, midface, jawline, and combinations thereof of a face.

The microchannels may further comprise a plurality of pointed branches thereon. The pointed branches may puncture the air pouch to release the air encapsulated and may puncture the pouch to release the at least one active ingredient, when the multi-layered sheet mask is pressed.

Facial skin cells may be dehydrated, especially when the stratum corneum is thinned. The second layer of the multi-layered sheet mask may be used to address the issue of dehydrated facial skin by provision of the at least one first active ingredient. When the skin cells are hydrated by the at least one first active ingredient, the water content within the cells may cause the cells to swell, thus causing the skin to appear bouncy and reflects light well.

The at least one first active ingredient in each of the at least one pouch of the second layer may be the same or different from each other.

The first active ingredient may be selected from the group consisting of anti-pigmentation agent, anti-ageing agent, anti-wrinkle agent, anti-acne agent, moisturizing agent, treatment agent, and mixtures thereof.

The at least one first active ingredient may further comprise a fragrance.

The at least one pouch in the second layer of the multi-layered sheet mask may have a volume in a range of about 1 ml to about 5 ml, about 2 ml to about 5 ml, about 3 ml to about 5 ml, about 4 ml to about 5 ml, about 1 ml to about 2 ml, about 1 ml to about 3 ml, about 1 ml to about 4 ml, about 2 ml to about 3 ml, about 2 ml to about 4 ml, or about 3 ml to about 4 ml.

Other active ingredients for enhancing the aesthetics of facial skin may be provided by the third layer of the multi-layered sheet mask by provision of the at least one second active ingredient. Where different active ingredients are used, this may provide more benefits to the recipient.

The third layer may be portioned into a number of parts, each part receiving or containing the at least one second active ingredient that may be the same or different from each other; and
wherein the second active ingredient may be selected from the group consisting of oil, chemical sunscreen, anti-pigmentation agent, anti-ageing agent, anti-wrinkle agent, anti-acne agent, moisturizing agent, treatment agent, and mixtures thereof.

The at least one second active ingredient may further comprise a fragrance.

The at least one second active ingredient in the third layer of the multi-layered sheet mask may have a volume in a range of about 1 ml to about 5 ml, about 2 ml to about 5 ml, about 3 ml to about 5 ml, about 4 ml to about 5 ml, about 1 ml to about 2 ml, about 1 ml to about 3 ml, about 1 ml to about 4 ml, about 2 ml to about 3 ml, about 2 ml to about 4 ml, or about 3 ml to about 4 ml.

The anti-pigmentation agent may be selected from the group consisting of N-acetyl glucosamine, hydroquinone, kojic acid, arbutin, resveratrol, tranexamic acid, niacinamide, liquorice extract, azelaic acid, and mixtures thereof.

The anti-ageing agent may be selected from the group consisting of azelaic acid, retinoic acid, retinol, hyaluronic acid, ascorbic acid, vitamin E, allantoin, bisabolol, and mixtures thereof.

The anti-acne agent may be selected from the group consisting of retinoic acid, retinol, topical antibiotics, tea tree oil, usnic acid, gluconolactone, Cannabis sativus, and mixtures thereof.

The moisturizing agent may be selected from the group consisting of water, carbomer, humectants, glycerin, hydroxyethylurea, acetamidoethoxyethanol betaine, inositol, taurine, emollients, isopropyl isostearate, isostearyl isostearate, C12-13 alkyl lactate, ceramide, pseudo-ceramide, cetyl PG hydroxyethyl palmitamide, ceramide II complex, niacinamide, Eucalyptus globulus leaf extract, cetearyl glucoside, PEG-100 stearate, distearyl dimethyl ammonium chloride, ceteareth 20, PEG-40 stearate, cetearyl alcohol, stearyl alcohol, cetyl alcohol, glyceryl stearate, xanthan gum, carbomer, acrylates/C10-30 alkyl acrylate crosspolymer, phenoxyethanol, benzyl alcohol, benzalkonium chloride, ethylhexyl glycerin, caprylyl glycol, hexanediol, pentylene glycol, glutamic acid, N.N-diacetic acid, sodium phytate, tetrasodium iminodisuccinate, and mixtures thereof. The C12-13 alkyl lactate may stimulate epidermal lipids production and provide skin hydration. The Eucalyptus globulus leaf extract may promote epidermal lipids production and sensitize the skin.

The treatment agent may be selected from the group consisting of ceramide, collagen, antioxidant, anti-inflammatory, and mixtures thereof.

The oil may be selected from the group consisting of coconut oil, olive oil, sunflower seed oil, shea butter oil, jojoba oil, almond oil, grapeseed oil, blackcurrent seed oil, chamomile oil, rosehip seed oil, argan oil, marula oil, tea tree oil, safflower seed oil, cedarwood oil, vetiver oil, neroli oil, helichrysum oil, facial oil, essential oil, vitamin E oil, and mixtures thereof.

The oil may have UV-screening function.

The oil may provide skin with moisture by locking in hydration, especially after a moisturizing agent is used in the at least one first active ingredient.

The chemical sunscreen may be selected from the group consisting of oxybenzone, avobenzone, octisalate, octocrylene, hornosalate, octinoxate, and mixtures thereof.

The air pouch in the second layer of the multi-layered sheet mask may lift the plurality of microchannels when the air pouch is inflated.

The height of the air pouch may be in a range of about 400 μm to about 500 μm, about 420 μm to about 500 μm, about 440 μm to about 500 μm, about 460 μm to about 500 μm, about 480 μm to about 500 μm, about 400 μm to about 420 μm, about 400 μm to about 440 μm, about 400 μm to about 460 μm, about 400 μm to about 480 μm, about 420 μm to about 440 μm, about 420 μm to about 460 μm, about 420 μm to about 480 μm, about 440 μm to about 460 μm, about 440 μm to about 480 μm, or about 460 μm to about 480 μm.

When the porous first layer of the multi-layered sheet mask is being applied for exfoliation, the microchannels may not penetrate the skin until the air pouch is fully deflated. Hence, the air pouch may provide cushion for the first and second layers of the multi-layered sheet mask from microchannels penetration. Air inside the air pouch may be released through a twist-and-turn valve to allow the microchannels to penetrate the skin. Advantageously, the twist-and-turn valve may avoid accidental puncturing of the air pouch when some force is applied on the multi-layered sheet mask, for instance during the exfoliation process when the porous first layer of the multi-layered sheet mask is in use.

The size of the air pouch is not limited and may vary depending on the therapeutic application of the multi-layered sheet mask.

The air pouch may be made from high molecular-weight polymer such as poly(lactic-co-glycolic acid) (PLGA), poly(L-lactic acid) (PLLA) or polyvinyl chloride (PVC). The high molecular weight polymer may be biodegradable or non-biodegradable.

The molecular weight of the high molecular weight polymer may be in the range of about g/mol to about 1,000,000 g/mol, about 100,000 g/mol to about 1,000,000 g/mol, about 200,000 g/mol to about 1,000,000 g/mol, about 500,000 g/mol to about 1,000,000 g/mol, about 800,000 g/mol to about 1,000,000 g/mol, about 80,000 g/mol to about 100,000 g/mol, about 80,000 g/mol to about 200,000 g/mol, about 80,000 g/mol to about 500,000 g/mol, about g/mol to about 800,000 g/mol, about 100,000 g/mol to about 200,000 g/mol, about 100,000 g/mol to about 500,000 g/mol, about 100,000 g/mol to about 800,000 g/mol, about 200,000 g/mol to about 500,000 g/mol, about 200,000 g/mol to about 800,000 g/mol, or about 500,000 g/mol to about 800,000 g/mol.

A circumferential edge comprising the circumferential edges of the porous first layer, the second layer, and the third layer, may have a width of at least about 5 mm.

A detachable fourth layer may also be incorporated into the multi-layered mask for final cleaning of the facial skin.

Exemplary, non-limiting embodiments of a mould for producing the three-dimensional patterns of the porous first layer of the multi-layered sheet mask will now be disclosed. The mould for producing the three-dimensional patterns of the porous first layer of the multi-layered sheet mask as disclosed herein comprises a negative replica of the three-dimensional patterns.

Advantageously, the mould may be designed to comprise a negative replica of the three-dimensional patterns of the porous first layer of the multi-layered sheet mask, which is dedicated for specific zones of the facial skin based on skin thickness and/or skin condition.

Further advantageously, the mould may allow the facile reproduction of complex micrometre-scale three-dimensional structures as exfoliators for cosmetic purposes.

When the polymer solution or melt forms the three-dimensional patterns in the mould, a sheet mask mould may be used to press the material forming the first layer onto the three-dimensional pattern mould. In this way, when the first layer material is removed from the three-dimensional pattern mould, the three-dimensional patterns will be formed (or stuck onto) the first later. Therefore, another mould such as a sheet mask mould can also be used.

The mould may further comprise a plurality of holes in sub-micrometre scale within the negative replica. The size range of these holes may be about 100 μm to about 1 mm.

The plurality of holes may allow negative pressure or air suctions to be applied on the mould during the process of casting when the polymer solution or melt is overlaid onto the mould, such that the polymer solution or melt may completely fill the contours of the mould.

The mould may be transparent. This may allow UV radiation to pass through from all sides of the mould to induce curing of the UV-curable polymer solution or melt that is overlaid onto the mould.

The mould may be formed by polymerizing a resin over a printed mould comprising a positive replica of the three-dimensional patterns. The printed mould may be a 3D-printed mould.

The mould may be formed in an inert gas environment (e.g., nitrogen gas or argon gas) with constant negative pressure applied, to avoid inhibition by the presence of oxygen during the polymerization process.

Exemplary, non-limiting embodiments of a method of producing a multi-layered sheet mask will now be disclosed.

The method of producing a multi-layered sheet mask comprises the steps of:
a) forming a porous first layer having a first side and a second side, by contacting a mask-shaped biocompatible material with a whole facial mask mould having a plurality of three-dimensional patterns thereon, wherein the three-dimensional patterns comprise a material selected from the group comprising of biocompatible material, exfoliating agent and combinations thereof, and drying for a duration;
b) attaching the second layer to the second side of the porous first layer by bonding the circumferential edges of the first and second layers together; and
c) attaching a third layer to the second layer by bonding the circumferential edges of the second and third layers together;
   wherein the multi-layered sheet mask comprises at least
a porous first layer wherein the first side is opposite to the second side and comprises a plurality of three-dimensional patterns extending therefrom;
a second layer attached to the second side of the porous first layer, the second layer comprising at least one pouch for receiving or containing at least one first active ingredient; a porous support structure; an air pouch that is sandwiched between the second side of the porous first layer and the porous support structure; and a plurality of microchannels that extend from the pouch and beyond the first side of the porous first layer when the air pouch is deflated; and
a third layer attached to the second layer for receiving or containing at least one second active ingredient.

Advantageously, the method may result in a porous first layer of multi-layered sheet mask which offers both mechanical and chemical exfoliation via the three-dimensional patters for effective removal of dead skin cells.

Further advantageously, the method may be adaptable to create additional layers, for example the fourth or fifth layers of mask, to offer additional cosmetic treatment for the facial skin. The method may also be adaptable to create multi-layered sheet mask for other parts of the body besides the face.

The three-dimensional patterns of step (a) may be formed by filling sections of the whole facial mask mould with the material of step (a) and vacuuming for a duration; and wherein the sections comprise of the mould as disclosed herein.

The sections may correspond to facial areas selected from the group consisting of hairline, forehead, temple, nose, cutaneous upper lip, philtum, philtum crest, cutaneous lower lip, chin, cheeks, midface, jawline, and combinations thereof.

The attaching of steps (b) and (b) may be done by heat bonding.

The attaching of step (b) may further comprise the step of applying fastening strips between the first and second layers; or wherein the attaching of step (c) may further comprise the step of applying fastening strips between the second and third layers.

The fastening strips may be Velcro strips, hooks or loops.

The use of heat bonding and/or Velcro strips may be safer and cost-efficient as compared to the use of adhesives.

The bonding of steps (b) and (c) may be for at least a width of about 5 mm of the circumferential edges. A broader width used may reduce the functional area of the multi-layered sheet mask, while a narrower width below about 5 mm may not result in stable attachment of the layers of the multi-layered sheet mask.

The porous first layer and the porous support structure of the second layer may comprise a plurality of slits. The method may thus further comprise the step of (d) forming slits in the porous first layer and the porous support structure. The forming step (d) may comprise the step of stamp cutting slits into the first layer or support structure. Alternatively, the forming step (d) may comprise the step of forming pores into the first layer or support structure that then form the slits.

### DESCRIPTION OF DRAWINGS

The accompanying drawings illustrate a disclosed embodiment and serve to explain the principles of the disclosed embodiment. It is to be understood, however, that the drawings are designed for purposes of illustration only, and not as a definition of the limits of the invention.

FIG. 1 is a schematic cross-sectional illustration of the multi-layered sheet mask 5000 applied on the skin 1 comprising dermis layer 2, epidermis layer 3 and stratum corneum 4, where the multi-layered sheet mask 5000 comprises the following components: porous first layer 5, three-dimensional patterns 6, second layer 7, air pouch 8, porous support structure 9, pouch 10 for receiving or containing at least one first active ingredient, microchannel 11, third layer 12, second active ingredient 13, and slits 14. The direction of flow of active ingredients as indicated by arrow 20 which is pointing from the mask 5000 towards the skin 1.

FIG. 2A is a schematic illustration of different zones A, B and C of a facial skin based on different skin thickness, where the microchannels of the second layer of the multi-layered sheet mask have different heights in corresponding to the skin thickness of each of these zones.

FIG. 2B is a schematic illustration of the foldable edges (dotted lines) of the multi-layered sheet mask.

FIG. 3A is a schematic illustration of an example of a design for the porous first layer of the multi-layered sheet mask that has various patterns of three-dimensional patterns at different regions corresponding to the facial skin.

FIG. 3B is schematic illustration of an example of a design for the porous first layer of the multi-layered sheet mask of FIG. 3A with a close-up view of the various patterns of three-dimensional patterns corresponding to different regions of the facial skin.

FIG. 4 is a schematic illustration of an example of a design for the second layer of the multi-layered sheet mask that has pouches positioned at different regions corresponding to different parts of the facial skin, each pouch 21 containing at least one first active ingredient and designed to be activated by one or more twist- and turn-valves 22. The darker regions in this figure (such as below the eyes area or above the lip area) correspond to the microchannel positions that extend across the first layer to illustrate the flow of the active ingredient(s) (such as serum) through the first layer and into the facial skin at the specific locations. For clarity, the darker regions are not represented by reference numerals 21 and 22 but are the darker regions (represented by the darker dots) in the other parts of the multi-layered sheet mask.

FIG. 5A is a drawing featuring an example of a 3D-printed mould of three-dimensional pattern with pointed three-dimensional shape comprising multiple spikes on two tiers, some spikes positioned at an angle from the vertical plane.

FIG. 5B is a drawing featuring an array design of the 3D-printed mould of three-dimensional pattern of FIG. 5A.

FIG. 5C is a microscopic side view of the 3D-printed mould of three-dimensional pattern of FIG. 5A.

FIG. 5D is a microscopic isometric view of the 3D-printed mould of three-dimensional pattern of FIG. 5A.

FIG. 5E is a microscopic top view of the 3D-printed mould of three-dimensional pattern of FIG. 5A.

FIG. 6A is a drawing featuring an example of a 3D-printed mould three-dimensional pattern with pointed three-dimensional shape comprising a cluster of multiple spikes on the centre of a hemisphere, some spikes positioned at an angle from the vertical plane.

FIG. 6B is a drawing featuring an array design of the 3D-printed mould of three-dimensional pattern of FIG. 6A.

FIG. 6C is a microscopic side view of the 3D-printed mould of three-dimensional pattern of FIG. 6A.

FIG. 6D is a microscopic isometric view of the 3D-printed mould of three-dimensional pattern of FIG. 6A.

FIG. 6E is a microscopic top view of the 3D-printed mould of three-dimensional pattern of FIG. 6A.

FIG. 7A is a drawing featuring an example of a 3D-printed mould of three-dimensional pattern with pointed three-dimensional shape comprising a spike on the centre of a hemisphere.

FIG. 7B is a drawing featuring an array design of the 3D-printed mould of three-dimensional pattern of FIG. 7A.

FIG. 7C is a microscopic side view of the 3D-printed mould of three-dimensional pattern of FIG. 7A.

FIG. 7D is a microscopic isometric view of the 3D-printed mould of three-dimensional pattern of FIG. 7A.

FIG. 7E is a microscopic top view of the 3D-printed mould of three-dimensional pattern of FIG. 7A.

FIG. 8 is a drawing featuring an array design of the 3D-printed mould of three-dimensional pattern with pointed three-dimensional shape comprising a partial irregular polyhedron.

FIG. 9 is a microscopic view of the three-dimensional patterns filled with hyaluronic acid as an exfoliating agent, wherein the three-dimensional patterns are based on pointed three-dimensional shape of (a) according to FIG. 5A, (b) according to FIG. 6A, (c) according to FIG. 7A, and (d) according to FIG. 8.

FIG. 10 is a schematic illustration of the first side 30 of the porous first layer 5 of the multi-layered sheet mask with three-dimensional patterns 6 that have different pointed three-dimensional shapes as skin-engaging ends, such as the stellated polyhedron 41, sphere and cones 42, and partial irregular polyhedron 43.

FIG. 11A is a schematic illustration of the first side 30 of the porous first layer 5 of the multi-layered sheet mask with three-dimensional patterns 6 having a diameter 51 and a height 52.

FIG. 11B is a schematic illustration showing some examples of three-dimensional patterns 6 with a diameter 51, a height 52, and a height of the corresponding pointed three-dimensional shape 53 of the three-dimensional pattern 6. The three-dimensional pattern may have various spike designs 60.

FIG. 12 is a schematic diagram illustrating the process of preparing a polymeric mould comprising a negative replica of the three-dimensional patterns (steps 1 and 2) and thereafter preparing a three-dimensional pattern which is a positive replica of the 3D-printed mould (steps 3 and 4).

FIG. 13 is a schematic diagram illustrating the general process of preparing the porous first layer of a multi-layered sheet mask.

FIG. 14 is a schematic diagram illustrating the process of preparing a whole facial mask which forms the complete porous first layer of a multi-layered sheet mask where the three-dimensional patterns in the porous first layer comprise an exfoliating agent.

FIG. 15 is a schematic diagram illustrating a whole facial mask mould design depicting (a) dimension of the whole facial mask, and (b) whole facial mask design with hollow areas as positions for mould comprising negative replica of three-dimensional patterns, wherein the whole facial mask forms the complete porous first layer of a multi-layered sheet mask.

FIG. 16 is a photo of a prototype 3D-printed whole facial mask with (200) hollow areas as positions for mould comprising negative replica of three-dimensional patterns, wherein the whole facial mask forms the complete porous first layer of a multi-layered sheet mask. This mould can be used as a sheet mask mould as mentioned above.

FIG. 17A is a photo of a prototype of a whole cellulose facial mask with three-dimensional patterns comprising hyaluronic acid, wherein the whole cellulose facial mask forms the complete porous first layer of a multi-layered sheet mask.

FIG. 17B is a photo of a prototype whole cellulose facial mask with three-dimensional patterns comprising hyaluronic acid at specific positions labelled as 300, 400, 500, 600, 700, 800 and 900, while the ring boundary 1000 prevents the leakage of active ingredients from the mask. The whole cellulose facial mask forms the complete porous first layer of a multi-layered sheet mask as defined herein. When used as a first layer of the multi-layered sheet mask, the three-dimensional patterns that comprises hyaluronic acid puncture the skin and thus improve permeation into the skin.

FIG. 18 shows the results of a skin test where (A) is a graph depicting the skin permeation of lidocaine over time and (B) is a chart depicting lidocaine in skin deposit after minutes, for skin treated with patches of four different designs of three-dimensional patterns. Here, the first layer was used for testing whereby all of the three-dimensional patterns were made of hyaluronic acid.

### DETAILED DESCRIPTION OF DRAWINGS

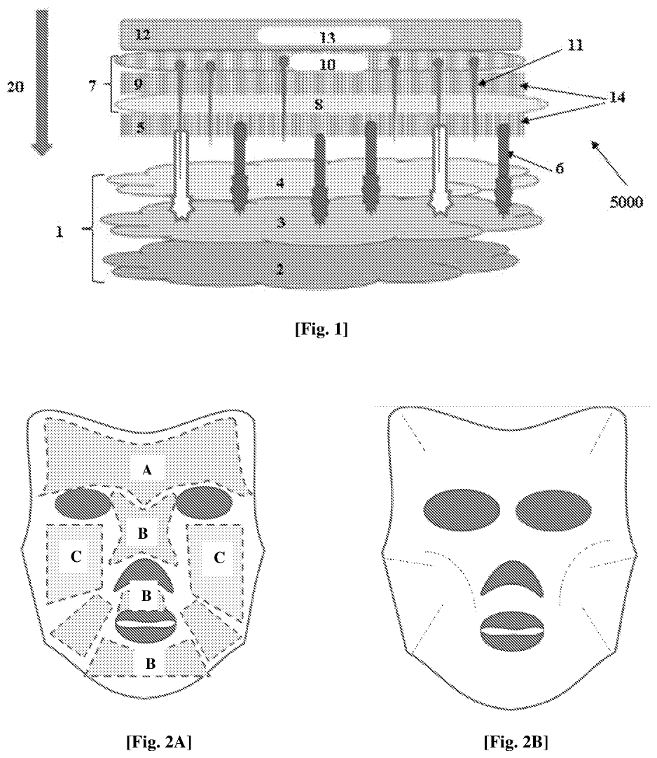

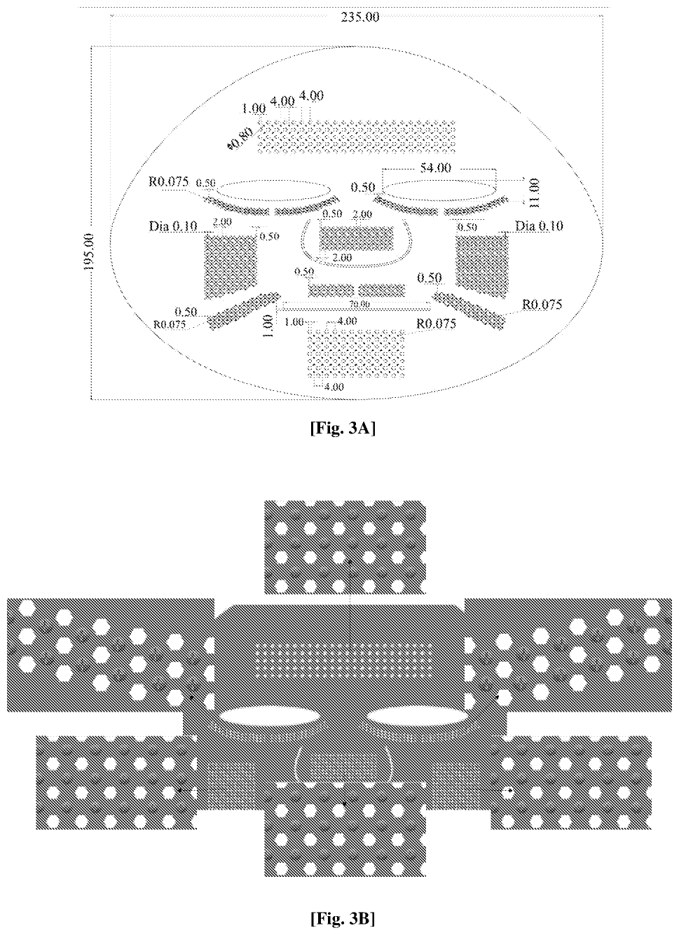

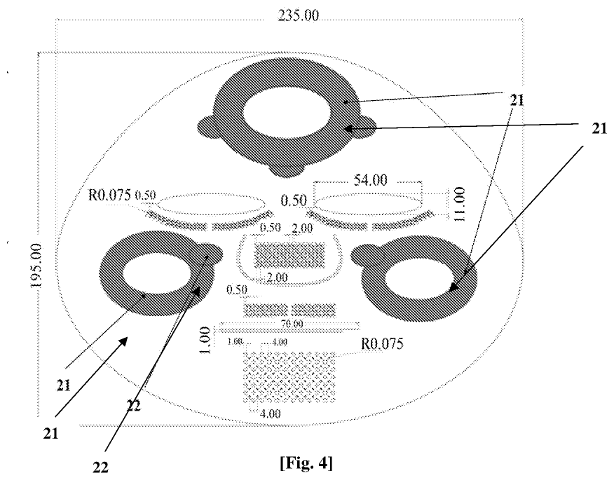

FIG. 1 is a schematic cross-sectional illustration of the multi-layered sheet mask 5000 applied on the skin 1 comprising dermis layer 2, epidermis layer 3 and stratum corneum 4. The porous first layer 5 of the multi-layered sheet mask 5000 has a plurality of three-dimensional patterns 6 thereon. These three-dimensional patterns 6 with pointed three-dimensional shapes penetrate through the stratum corneum 4 during exfoliation. Some of the three-dimensional patterns 6 pass through the stratum corneum 4 and some of three-dimensional pattern 6 reach the dermis layer 2. The three-dimensional patterns 6 may be inserted from within the porous first layer 5 of the multi-layered sheet mask 5000 or may be formed on the surface of the first side of the porous first layer 5 of the multi-layered sheet mask 5000. Slits 14 are arranged within the support structure 9 of the second layer 7, as well as within the porous first layer 5 to allow the first active ingredient to flow from the pouch 10 in the second layer 7 to the surface of the skin 4 when the first active ingredient is released to flow from the pouch 10 by activating a twist-and-turn valve on the pouch 10 or when the pouch 10 is punctured by the microchannels 11. The slits 14 may have a hexagonal prism shape. The second layer 7 of the multi-layered sheet mask 5000 incorporates at least a pouch 10 for receiving or containing a first active ingredient and an air pouch 8. Each of the microchannels 11 has two ends, where one end is inserted into the pouch 10 and the other end comprising a pointed three-dimensional shape which penetrates the dermis layer 2 of the skin when the air pouch 8 is deflated. The three-dimensional patterns 6 may have a hollow center for the pointed microchannel 11 to penetrate through. The third layer 12 of the multi-layered sheet mask 5000 incorporates a second active ingredient 13 for cosmetic enhancement as needed. For instance, the third layer 12 may be portioned into a number of parts that contain chemical sunscreen or oil. Alternatively, the third layer 12 may have the second active ingredient 13 encapsulated in a pouch to be released by activating a twist-and-turn valve in the pouch. The second active ingredient 13 will pass through the second layer 7 and the porous first layer 5 to the skin 1 through the slits 14. The direction of flow of active ingredients is indicated by arrow 20. Optionally, a detachable fourth layer (not shown in FIG. 1) may be incorporated above the third layer 12 to provide further cosmetic functions such as final cleansing of the facial surface.

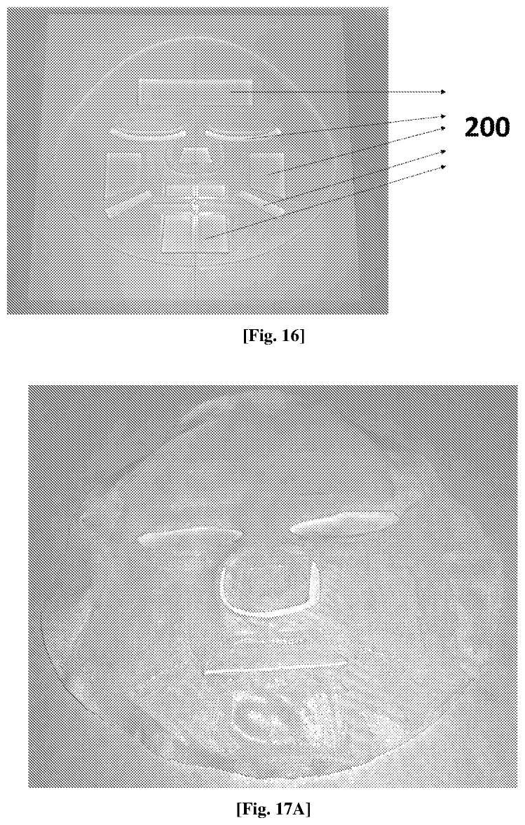

FIG. 2A is a schematic illustration of different zones A, B and C of a facial skin based on different skin thickness, where the microchannels of the second layer of the multi-layered sheet mask have different heights corresponding to each of these zones. When the microchannels are used in combination with the three-dimensional patterns of the multi-layered sheet mask, for instance when the air pouch in the second layer is deflated, some of the microchannels penetrate the three-dimensional patterns into the skin or directly into the skin to deliver a portion of the first active ingredient (e.g., collagen or moisturizer), while another portion of first active ingredient flows through the slits in the second and first layers to reach the skin. The type and volume of first active ingredient to be supplied to each of the zone may be arranged by tailoring the contents of the pouch or pouches in the second layer of the multi-layered sheet mask. Hence, exfoliation and stimulation of collagen formation or hydration can be performed on a facial skin of varying skin thickness in different zones to achieve a more targeted treatment. As an example, the depth of penetration in each zone is as follows: zone A—1500 to 1800 μm; zone B—1200 to 1500 μm; and zone C—1600 to 1900 μm. Additionally, when the mould of FIG. 16 is used as a sheet mask mould, polymeric moulds that are used to form the three-dimensional patterns can be inserted into each of the voids in regions A, B and C in FIG. 2A and a solution material for the three-dimensional patterns filled into the polymeric moulds. Thereafter, a mask-shaped biocompatible material, such as cellulose, can be pressed onto the polymeric moulds using the sheet mask mould of FIG. 16.

FIG. 4 is a schematic illustration of an example of a design for the second layer of the multi-layered sheet mask that has pouches positioned at different regions corresponding to different parts of the facial skin, each pouch 21 containing at least one first active ingredient and designed to be activated by one or more twist-and-turn valves 22. For example, the pouch 21 at the forehead region has a diameter of about 70 mm by about 60 mm, and each of the three corresponding twist-and-turn valves 22 for at least one first active ingredient elution have a diameter of about 10 mm. Each of the pouch 21 at the cheeks have a diameter of about 50 mm by about 40 mm, and the corresponding twist-and-turn valve 22 for at least one first active ingredient elution have a diameter of about 10 mm. The volume of pouch in each zone is about 1 ml to 50 ml.

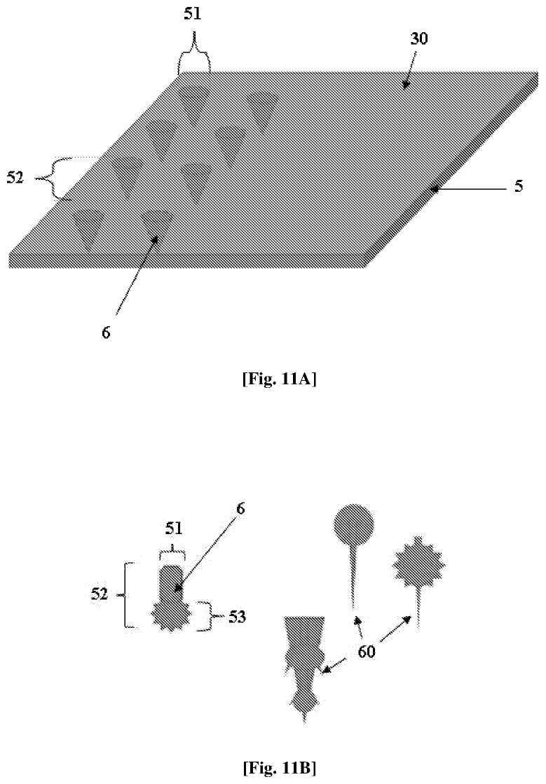

FIG. 11A is a schematic illustration of the first side 30 of the porous first layer 5 of the multi-layered sheet mask with three-dimensional patterns 6 that have pointed three-dimensional shape in the shape of cones, having a diameter 51 and a height 52. The three-dimensional patterns 6 may have different heights in order to penetrate different depths of facial skin.

FIG. 11B is a schematic illustration showing some examples three-dimensional patterns 6, with a diameter 51, a height 52 and a height of the corresponding pointed three-dimensional shape 53 of the three-dimensional pattern 6, The three-dimensional patterns 6 can have various spike designs 60 which can be positioned vertically on a three-dimensional pattern or positioned at an angle from the vertical plane on a three-dimensional pattern. The three-dimensional patterns 6 and the pointed three-dimensional shape 53 may have different heights in order to penetrate different depths of facial skin. Further, the angle and direction of the spike(s) 60 may also vary for different skin thickness and skin condition to achieve different treatment effects. As mentioned above, where the spike is positioned at an angle from the vertical plane, the spike may have higher penetration area from the angle and from an adjacent area of a center spike, as compared to a spike that is not positioned at an angle from the vertical plane. Additionally, when the spike is positioned at an angle from the vertical plane, the spike may have a different depth of penetration when the first layer of the multi-layered sheet mask is compressed, as compared to a spike that is not positioned at an angle.

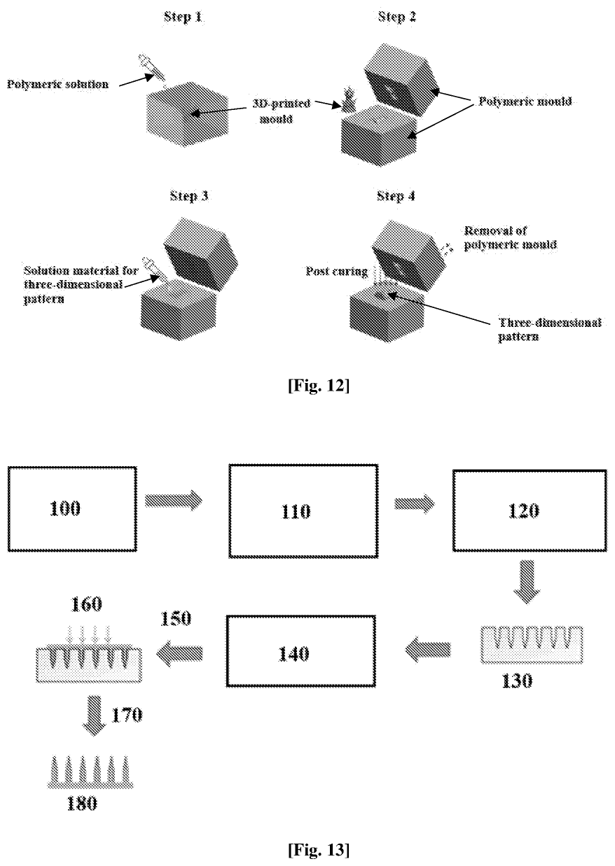

FIG. 12 is a schematic diagram illustrating the process of preparing a polymeric mould comprising a negative replica of the desired three-dimensional pattern for the porous first layer of the multi-layered sheet mask (i.e., steps 1 and 2) and thereafter preparing a three-dimensional pattern which is a positive replica of the 3D-printed mould (i.e., steps 3 and 4). For instance, step 1 involves casting and curing a polymeric material, such as polydimethylsiloxane (PDMS), on a 3D-printed mould, and step 2 involves removing the cured polymeric mould from the 3D-printed mould. The polymeric mould thus obtained comprises a negative replica of the desired three-dimensional pattern. Thereafter, in step 3, a solution material for the three-dimensional pattern is filled into the polymeric mould obtained from step 2. Thereafter, in step 4, the three-dimensional pattern is subjected to curing, removal of a portion of the polymeric mould to expose the three-dimensional pattern formed within, and post-curing the three-dimensional pattern at an elevated temperature for a duration. The three-dimensional pattern thus obtained may be incorporated into the porous first layer of the multi-layered sheet mask such that the three-dimensional patterns function as a mechanical exfoliator (rather than comprising the exfoliating agent therein).

FIG. 13 is a schematic diagram illustrating the general process of preparing the porous first layer of a multi-layered sheet mask. Firstly, step 100 involves preparing a 3D-printed solid master mould with positive replica of desired three-dimensional patterns. The solid master mould may be made of steel. Thereafter, step 110 involves casting a polymeric material such as polydimethylsiloxane (PDMS) over the 3D-printed solid master mould. Step 120 involves curing the PDMS polymer on the master mould and ejecting the cured polymer to obtain a polymeric mould 130 with negative replica of the three-dimensional patterns. Next, step 140 involves casting a biocompatible material which is a polymer solution or melt, for example gelatin methacrylate (GelMA), over the polymeric mould, applying vacuum in step 150. The polymeric mould may comprise a plurality of holes in sub-micrometre scale within the negative replica, such that when vacuum is applied in step 150, air suction causes the biocompatible material in a solution or melt form to fill the entire contours of the polymeric mould 130 to effectively assume the positive replica shape of the three-dimensional patterns. Thereafter, UV radiation is applied in step 160 to cure the biocompatible material, followed by peeling off in step 170 to obtain the formed three-dimensional patterns 180. The polymeric mould 130 may also be transparent and the environment in step 160 may be filled with inert gas such as nitrogen or argon, with continuous vacuum applied such that UV radiation in step 160 can penetrate all sides of the polymeric mould to induce curing of the biocompatible material. The step of 160 will not be inhibited by oxygen for radical polymerization if the solution formulation for the biocompatible material is UV curable. The biocompatible material may be woven or non-woven and alternatively, the biocompatible material may be hot pressed on the polymeric mould to form the three-dimensional patterns 180 instead of undergoing steps 140, 150 and 160.

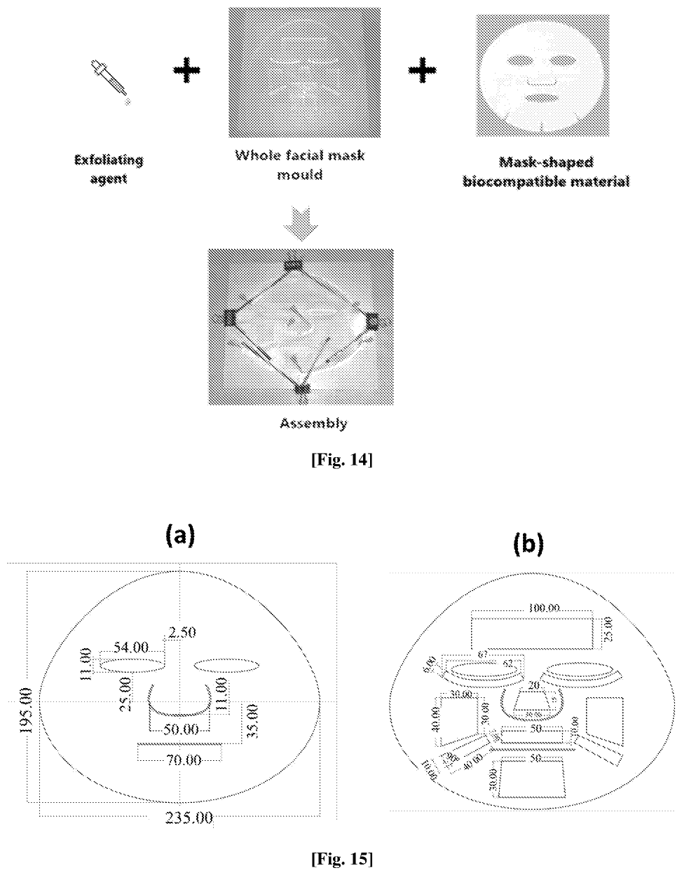

FIG. 14 is a schematic diagram illustrating the process of preparing a whole facial mask which forms the complete porous first layer of a multi-layered sheet mask. A solution of hyaluronic acid is filled in the sections of the whole facial mask mould comprising three-dimensional patterns to form three-dimensional patterns comprising hyaluronic acid as exfoliating agent. Thereafter, the whole facial mask mould is brought into contact with a mask-shaped biocompatible material, such as cellulose, and secured using stainless steel rods and needle heads to form an assembly to ensure adequate contact between the mask-shaped biocompatible material and the hyaluronic acid. The formed whole facial mask is removed from the whole facial mask mould after the hyaluronic acid solution has completely dried.

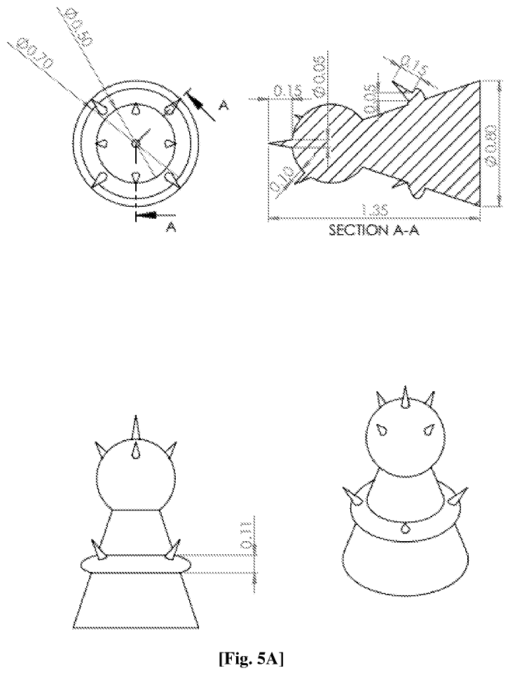

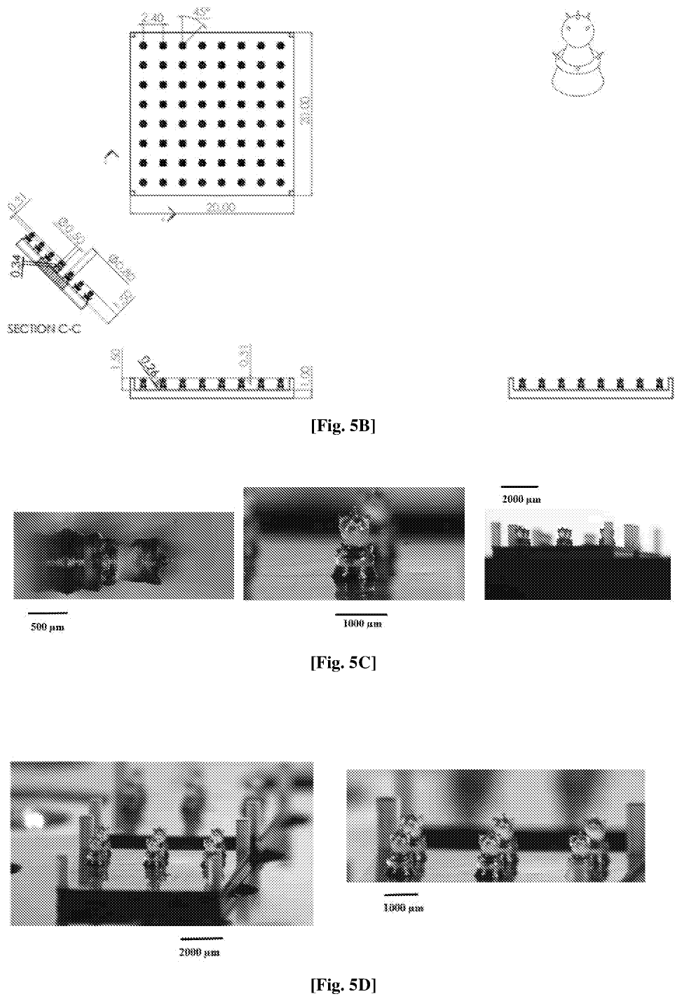

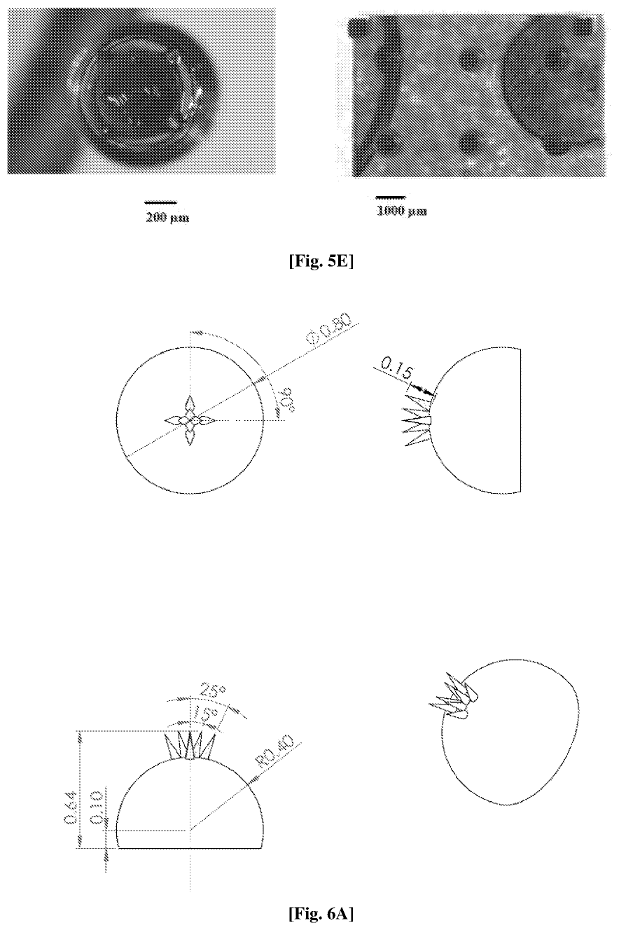

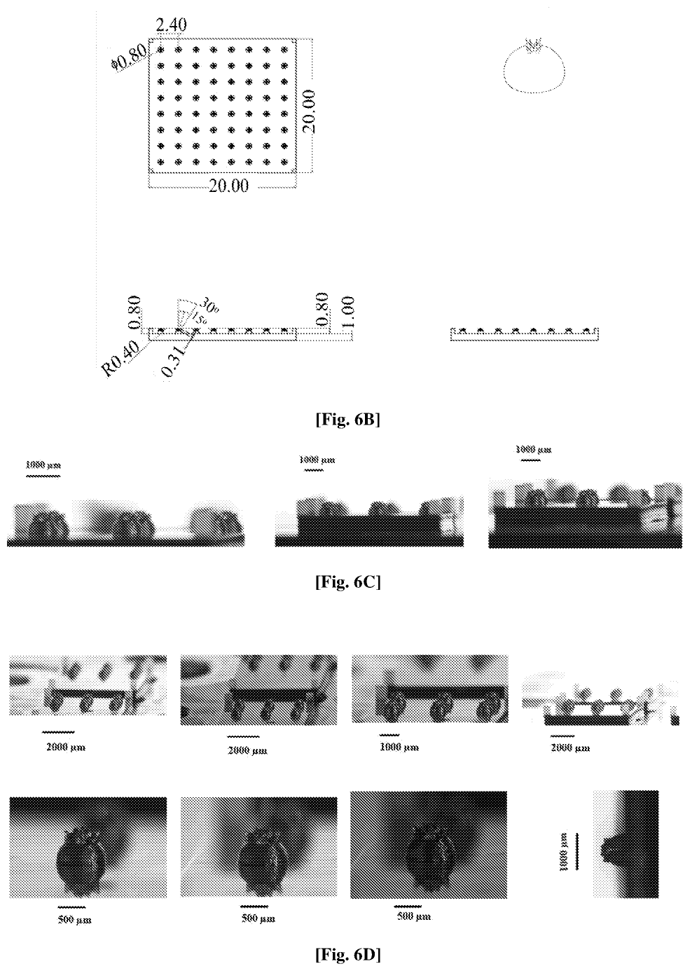

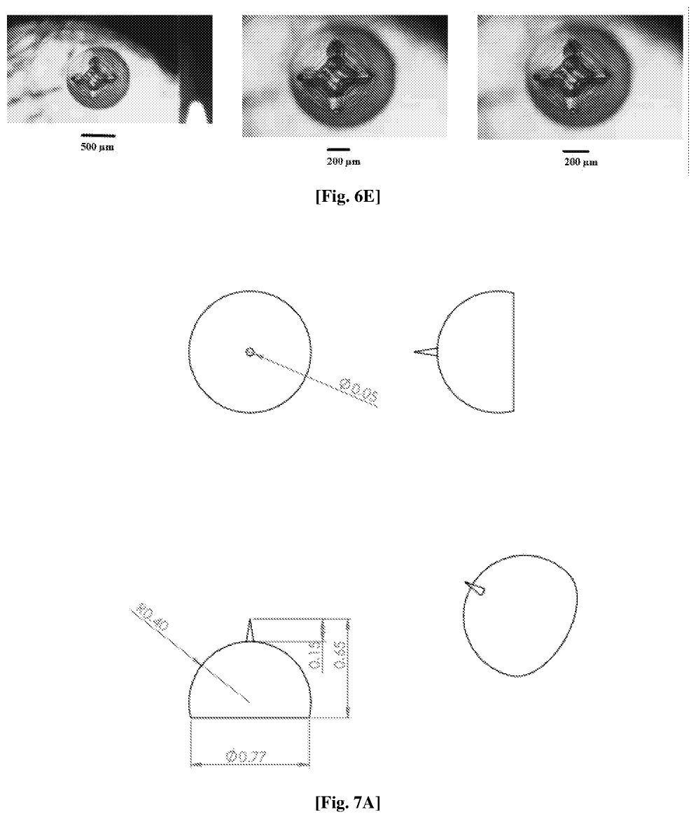

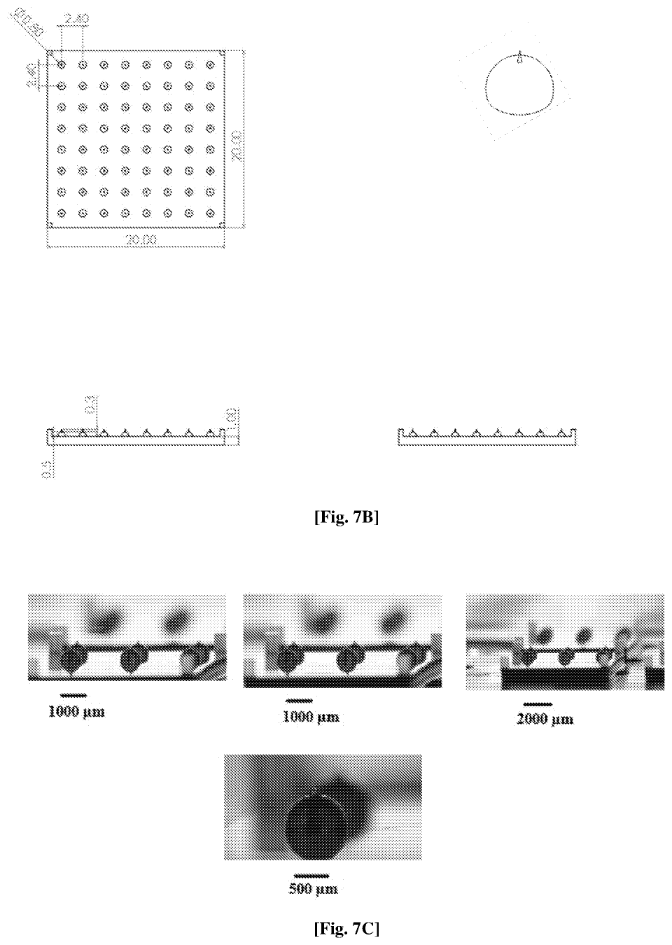

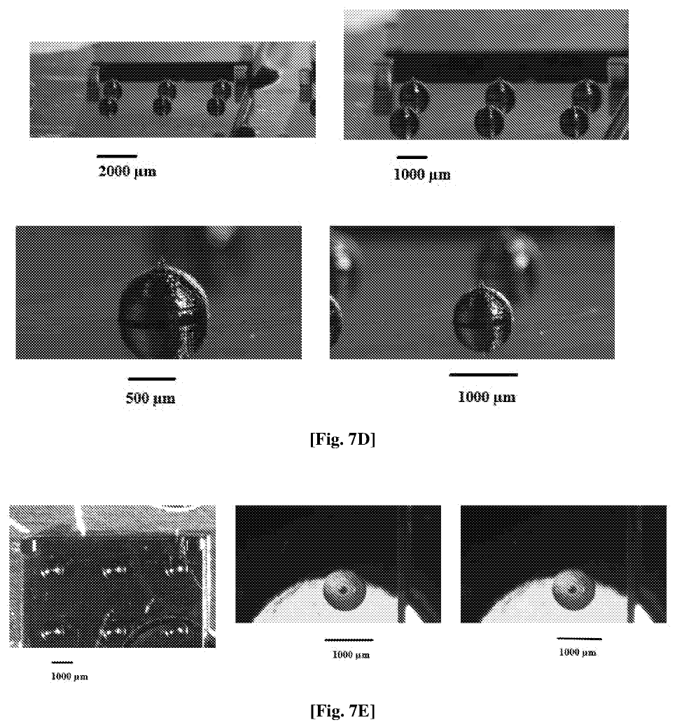

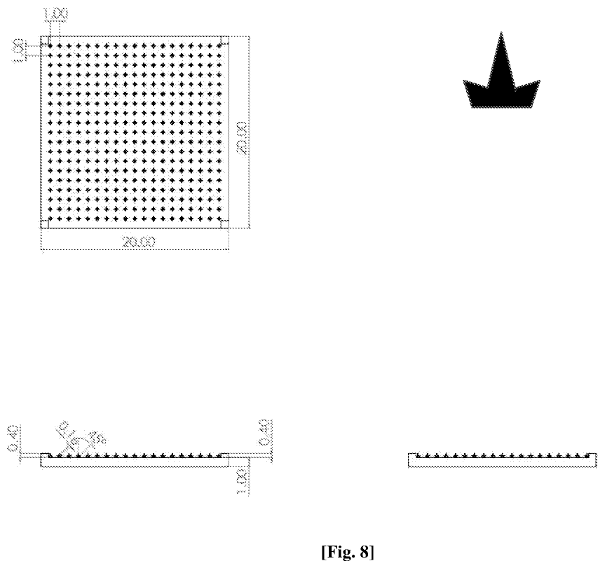

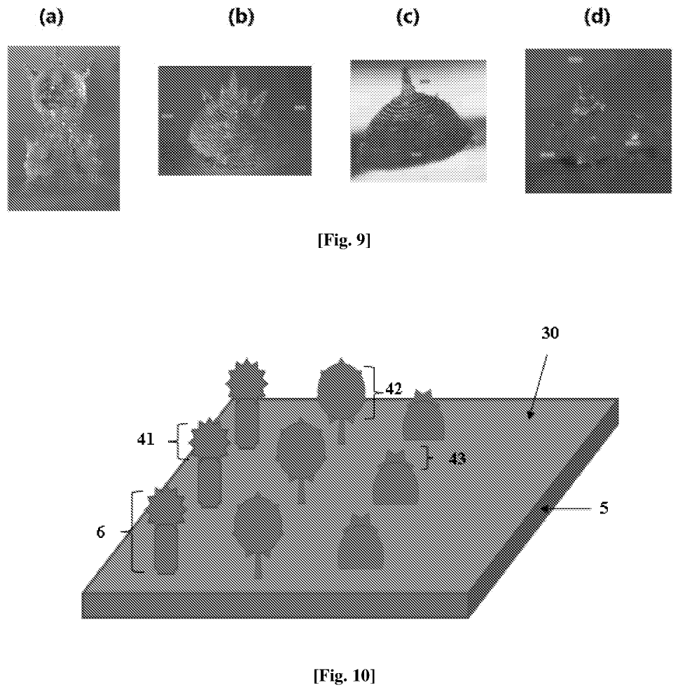

FIG. 18 shows the results of a skin test where (A) is a graph depicting the skin permeation of lidocaine over time and (B) is a chart depicting lidocaine in skin deposit after 60 minutes, for skin treated with patches of four different designs of three-dimensional patterns as compared to control which is a cream applied to the skin directly without any treatment. Design 1 to 4 correspond to the three-dimensional patterns shown in FIG. 5A, FIG. 6A, FIG. 7A and FIG. 8 respectively. Design 1 showed the highest lidocaine absorption and had the highest lidocaine skin deposits among the four designs.

### EXAMPLES

Non-limiting examples of the invention will be further described in greater details by reference to specific Examples, which should not be construed as in any way limiting the scope of the invention.

### Example 1: Synthesis of 3D-Printed Moulds

Models of the 3D-printed moulds comprising positive replicas of the desired three-dimensional patterns were designed using SolidWorks (Dassult Systèmes, USA) to have various pointed three-dimensional shapes and synthesized by 3D printing using a Titan 2HR 3D printer (Kudo 3D, USA) with Optoma Projector with light spectrum >=400 nm (visible range) as the light source, light intensity set at 3000 lumens, slicing software from Creation workshop, resin (3DM cast from ADMAT of France), layer splicing at 25 μm, pixel resolution at 26 μm and exposure time set at 6 to 16 seconds.

### Example 2: Synthesis of Polymeric Mould and Three-Dimensional Patterns

With reference to FIG. 12, FIG. 12 provides the steps used to make the mould and to generate the three-dimensional patterns.

### Materials

Polydimethylsiloxane (PDMS) is a two-part polymer (Base Elastomer and Curing Agent). Here, Sylgard 184 from Dow Corning was used. The standard mixing ratio for PDMS is 10-parts base elastomer and 1-part curing agent. This ratio provides the mechanical properties that are desirable and optimum biocompatibility. The solution material (or precursor solution) for three-dimensional pattern can be any biocompatible material but in this example, hyaluronic acid was used as the solution material (or precursor solution).

### Procedure

Firstly, to prepare the polymeric solution, the base elastomer and curing agent of the PDMS were mixed at a ratio recommended by the manufacturer which is a base to curing agent ratio of 10:1 (by parts) and de-aired for 30 minutes by applying negative pressure of −95 kPa. The polymeric solution was poured onto the cured 3D-printed mould in a container and vacuumed at −95 kPa for 30 minutes to ensure that bubbles were removed completely. The container containing the polymeric solution was cured at 80° C. for 30 minutes. After the polymeric solution was completely cured, the 3D-printed mould was removed from the cured polymeric mould by applying a force to detach the 3D-printed mould from the cured polymeric mould (which is a polydimethylsiloxane mold). This is a pre-casting step to create two halves of the negative replica of the 3D-printed mold.

Thereafter, hyaluronic acid (4 g of HA in 20 ml DI water for 20% stock HA solution) was filled into the microneedles of the polymeric PDMS mould and the whole 3D printed PLA mask mold was put under vacuum at 95 kPa for 30 minutes. The cellulose mask sheet was placed on top of the PLA mould with PDMS negative needles filled with hyaluronic in the PDMS mould and ensuring the cellulose sheet is in contact with the hyaluronic acid three-dimensional patterns. A weight was placed and secured with stainless steel rods to ensure good contact between the mask and the filled casted HA solution in the PDMS mould. The cellulose mask with filled HA solution was left dried at room conditions for at least 48 hours. After the HA solution was completely dried out, the cellulose mask with the HA three0dimensional patterns was removed from the mould, thus forming the first layer of the multi-layered sheet mask.

### Example 3: Synthesis of Prototype Polymeric Mould, Prototype Whole Facial Mask Mould and Porous First Layer of Multi-Layered Sheet Mask

### Materials

The following materials were purchased commercially for use in this example without modification: hyaluronic acid molecular weight 8 to 15 kDa (MakingCosmetics, USA), Sylgard 184 silicon elastomer kit (Dow Corning, USA), polylactic acid (PLA, Makerbot Industries, USA), 3DM-CAST resin (ADMAT, France) and mask-shaped cellulose (under brand name BIO-Celtox™ obtained from Guangzhou Yurui Cosmetics Co., Ltd, China) and isopropyl alcohol. De-ionised water was used.

### Procedure

### Digital Light Processing (DLP) Printing Method

Titan 2HR 3D printer (Kudo 3D, USA) was used to print the 3D-printed mould of the three-dimensional patterns. The three-dimensional patterns may be specific to a certain zone on the facial skin, as shown in FIG. 15B. The Titan 2HR 3D printer uses optoma projector with light spectrum ≥400 nm wavelength (visible range) as its light source. A model of the 3D-printed mould was designed using SolidWorks (Dassult Systèmes, USA) and sliced using Creation Workshop (XAYAV, USA). The 3D printer was calibrated prior to printing. Sliced STL files of the model of the 3D-printed mould were uploaded to the 3D printer and printing parameters settings are as shown in Table 1.

TABLE 1
Printing Parameters of DLP Printing Method

| Parameter | Value | |
|---|---|---|
| Light intensity | 3000 | lumens |
| Layer Slicing | 25 | μm |
| Pixel resolution | 26 | μm |
| Exposure time | 6 to 16 | seconds |

The 3D-printed mould was washed two times at 15 minutes per wash using isopropyl alcohol. Thereafter, the 3D-printed mould was air-dried at room temperature followed by post-curing under UV irradiation for 30 minutes. Multiple 3D-printed moulds may be synthesized for different zones of facial skin. Examples of the 3D-printed moulds of three-dimensional patterns are shown in FIG. 5A to FIG. 8.

### Casting of Polymeric Solution

A schematic diagram illustrating this process is shown in FIG. 12. Firstly, to prepare the polymeric solution, silicone elastomer was mixed at a ratio recommended by the manufacturer at base to curing agent ratio of 10:1 (by weight) and de-aired for 30 minutes by applying negative pressure of −95 kPa. The polymeric solution was poured onto the cured 3D-printed mould in a container and vacuumed at −95 kPa for 30 minutes. The container was placed in an oven at 80° C. for 2 hours for curing. After the polymeric solution was completely cured, the 3D-printed mould was removed from the cured polymeric mould. The cured prototype polymeric mould was ready for precursor solution filling. As the prototype polymeric mould may be specific to a certain zone on facial skin, multiple prototype polymeric moulds may be synthesized before assembly with a whole facial mask mould.

### Fused Deposition Modeling (FDM) Printing Method

MakerBot Replicator Z18 (Makerbot Industries, USA) and biodegradable polylactic acid (PLA, Makerbot Industries, USA) were used to make the prototype whole facial mask mould. A model of the whole facial mask mould was designed using SolidWorks (Dassult Systèmes, USA) according to the printing parameters settings shown in Table 2. The whole facial mask mould was designed with hollow portions on the forehead, under eyelids, cheeks, nose, smile lines, upper lip lines and chin as shown in FIG. 16, as these facial areas are to be filled with the prototype polymeric mould prepared as disclosed above, such that these facial areas would have their corresponding three-dimensional patterns in the whole facial mask mould. The polymeric mould was slotted inside designated holes on the whole facial mask mould. Alternatively, the whole facial mask mould as shown in FIG. 16 may be used as a sheet mask mould to contact a first layer material onto three-dimensional patterns when into onto their respective three-dimensional moulds.

TABLE 2
Printing Parameters of FDM Printing Method

| Parameter | Value | |
|---|---|---|
| Layer height | 200 | μm |
| Infill | 10% | |
| Material | Biodegradable polylactic acid | |
| Extruder parameter | 215° | C. |
| Print bed temperature | 90° | C. |

### Preparation of Whole Facial Mask Comprising an Exfoliating Agent

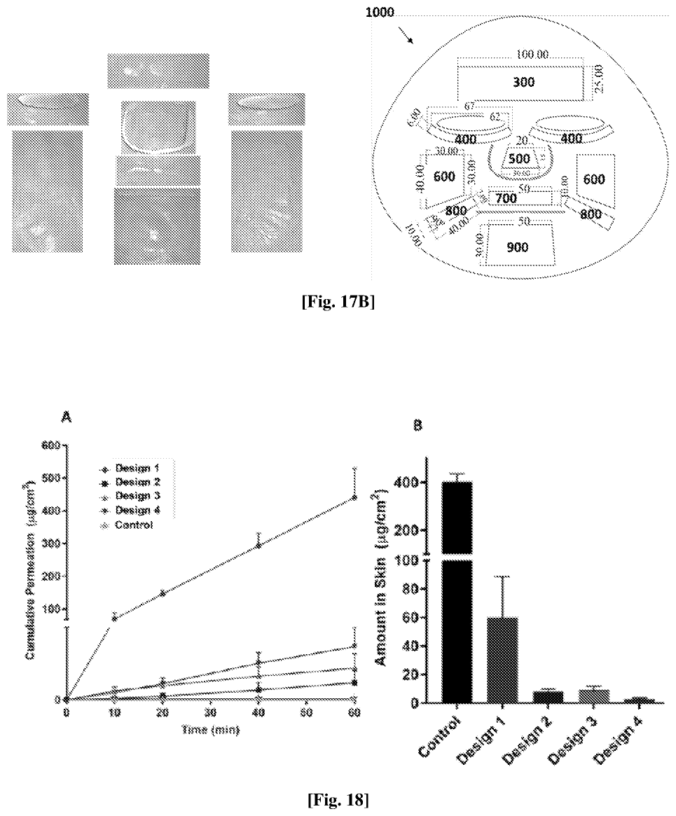

A schematic diagram illustrating the process to prepare a whole facial mask which forms the porous first layer of a multi-layered sheet mask, where the three-dimensional patterns comprise an exfoliating agent is shown in FIG. 14. Firstly, 4 g of hyaluronic acid was dissolved in 20 mL deionised water to prepare a stock hyaluronic acid solution. The slits of the porous first layer and slits of the porous support structure of the second layer can be formed by integrating honey comb protrusion on negative PDMS mold. The HA solution will be casted on the honey comb protruded structure PDMS mold. The hyaluronic acid solution was filled only in the areas of the whole facial mask mould that had the negative replica of the three-dimensional patterns (i.e., areas covered by the prototype polymeric moulds), and the whole facial mask mould was vacuumed at −95 kPa for 30 minutes. Thereafter, the whole facial mask mould was brought into contact with a mask-shaped cellulose and secured using stainless steel rods and needle heads to form an assembly to ensure adequate contact between the mask-shaped cellulose and the casted hyaluronic acid solution. The assembly was left to dry at room temperature for 2 days. After the casted hyaluronic acid solution was completely dried, the mask-shaped cellulose comprising hyaluronic acid three-dimensional patterns was removed from the whole facial mask mould. A photo of the whole cellulose facial mask comprising hyaluronic acid three-dimensional patterns is as shown in FIG. 17A, and a breakdown of the various parts of the whole cellulose facial mask is as shown in FIG. 17B.

### Example 4: Skin Test

### Materials

The following materials were purchased commercially for use in this example without modification: lidocaine, diclofenac sodium, acetonitrile HPLC grade (Merck, USA), ethanol, ammonium formate (Merck, USA), phosphate buffered saline (PBS, Vivantis, Singapore). Water used was purified by Mili-Q system. Human cadaver dermatone skin used for the analysis was obtained from Science Care (Phoenix, AZ, USA). The experiment was conducted using vertical Franz diffusion cells at 32° C. with an effective exposed area of 1 cm². The skin piece used in this experiment had a thickness of 150 μm to 200 μm and area of 2×2 cm.

### Procedure

An active ingredient composition used in this experiment comprised 23% lidocaine and 1% diclofenac sodium in the form of a cream. 5 mL of phosphate buffered saline (PBS) in the receptor compartment was used as the medium for absorption of the active ingredient composition. The test was conducted by applying patches containing three-dimensional patterns according to designs of FIG. 5A, FIG. 6A, FIG. 7A and FIG. 8 on the skin as treatment, before applying the active ingredient composition on the treated skin. For control, the skin was not treated with any patch. Thereafter, 1 mL samples were collected from the receptor medium after a time of 10 minutes, 20 minutes, 40 minutes, and 60 minutes and the same amount was replaced with fresh PBS. After 60 minutes of sample collection from the receptor medium, the rest of the receptor medium was discarded, the cream on the skin was removed and the skin was washed with PBS to remove residual cream. Washed skin was dispersed in 70% ethanol for 24 hours to extract the absorbed lidocaine content. All samples were analysed using a validated high pressure liquid chromatography (HPLC) method as shown in Table 3.

TABLE 3
Validated HPLC Method

| | |
|---|---|
| Instrument | Shimadzu LC-20AD with SDP-M20A UV detector (from Shimadzu |
| | Corporation, Japan) |
| Column | C18 |
| UV detection | 225 nm |
| Mobile phase | Organic phase (acetonitrile), aqueous phase (50 mM ammonium |
| composition | formate) |
| Program | Gradient flow |
| | At 0 minute 80% 50 mM ammonium formate, 20% acetonitrile |
| | To 9 minute 30% 50 mM ammonium formate, 70% acetonitrile; |
| | At 9.01 minute 80% 50 mM ammonium, 20% acetonitrile; |
| | To 12 minute 80% 50 mM ammonium formate, 20% acetonitrile |
| Flow rate | 0.8 mL/minute |
| Injection volume | 20 μL |

Based on the results in FIG. 18A, treatment of the skin with patches containing three-dimensional patterns increased the skin absorption of lidocaine at all time points compared to the control which did not receive treatment. However, the extent of absorption of lidocaine varied according to different designs of three-dimensional patterns. Design 1 showing the highest lidocaine absorption corresponds to the three-dimensional pattern with pointed three-dimensional shape comprising multiple spikes on two tiers, some spikes positioned at an angle from the vertical plane as in FIG. 5A. Further, based on the results of FIG. 18B, Design 1 also had the highest lidocaine skins deposits compared to other designs. However, the lidocaine deposits were highest for the control. The high amount of lidocaine deposits in the skin while having low lidocaine absorption for the untreated skin suggests that the lidocaine in the untreated skin only remains in the stratum corneum layer of the skin. In contrast, lidocaine deposits in the treated skin were much lower but had higher lidocaine absorption. Further, an un-quantifiable amount of lidocaine may be metabolized in the skin, thus converting the lidocaine to metabolites that were not detected. The lidocaine skin deposits that were quantified corresponds to unmetabolized lidocaine in the skin.

### INDUSTRIAL APPLICABILITY

The multi-layered sheet mask as disclosed herein may be used as a skincare product or a skin treatment product for providing a variety of cosmetic enhancements for facial skin, such as pigmentation alleviation, anti-wrinkle, anti-ageing, anti-acne, collagen growth stimulation and hydration. The multi-layered sheet mask may also be applied to other skin areas on the body besides facial skin.

The mould as disclosed herein may be used in the cosmetics, personal care and biomedical industries to produce complex three-dimensional patterns for enhanced skin engagement. The method as disclosed herein may be used in the cosmetics, personal care and biomedical industries to produce multi-layered structures that combine different functionalities of each layer into a single structure, with enhanced delivery of active ingredients into skin.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A multi-layered sheet mask comprising at least
a) a porous first layer having a first side and a second side, wherein the first side is opposite to the second side, and comprises a plurality of three-dimensional patterns extending therefrom;
b) a second layer attached to the second side of the porous first layer, the second layer comprising at least one pouch for receiving or containing at least one first active ingredient; a porous support structure; an air pouch that is sandwiched between the second side of the porous first layer and the porous support structure; and a plurality of microchannels that extend from the pouch and beyond the first side of the porous first layer when the air pouch is deflated; and
c) a third layer attached to the second layer for receiving or containing at least one second active ingredient.

2. The multi-layered sheet mask according to claim 1, wherein the porous first layer and the porous support structure of the second layer comprise a plurality of slits.

3. The multi-layered sheet mask according to claim 1, wherein the porous first layer and the three-dimensional patterns are made of a material independently selected from the group consisting of synthetic, regenerated and natural biocompatible materials.

4. The multi-layered sheet mask according to claim 3, wherein the material is selected from the group consisting of UV-curable polymer, UV-LED curable polymer, bioabsorbable polymer, cotton, nylon, nylon microfibre, regenerated cellulose fibre, biocellulose, foil, hyaluronic acid, and hydrogel.

5. The multi-layered sheet mask according to claim 1,
wherein the three-dimensional patterns comprise an exfoliating agent selected from the group consisting of alpha hydroxy acids, beta hydroxy acids, plant-based enzymes, animal-based enzymes and mixtures thereof; or
wherein the three-dimensional patterns comprise an exfoliating agent selected from the group consisting of lactic acid, lactobionic acid, glycolic acid, hydroxycaproic acid, hydroxycaprylic acid, citric acid, malic acid, mandelic acid, tartaric acid, phytic acid, salicylic acid, hyaluronic acid, azelaic acid, kojic acid, ascorbic acid, trichloroacetic acid, alguronic acid, lipoic acid, ferulic acid and mixtures thereof; or
wherein the three-dimensional patterns have an average diameter in a range of 1 μm to 1 mm, and a height in the range of 1 mm to 3 mm.

6. (canceled)

7. The multi-layered sheet mask according to claim 1, wherein the three-dimensional patterns comprise a plurality of pointed three-dimensional shapes as skin-engaging ends, wherein the pointed three-dimensional shapes are selected from the group consisting of cone, pyramid, stellated polyhedron, regular polyhedron, irregular polyhedron partial regular polyhedron, partial irregular polyhedron, sphere and at least one cone, sphere and at least one spike, sphere and at least one pyramid, hemisphere and at least one spike, spike, and combinations thereof.

8. The multi-layered sheet mask according to claim 5, wherein the pointed three-dimensional shapes have a height in a range of 200 μm to 250 μm.

9. The multi-layered sheet mask according to claim 7, wherein when the pointed three-dimensional shapes comprise of at least one spike, each spike has a length in a range of 40 μm to 100 μm.

10. The multi-layered sheet mask according to claim 9, wherein the spike is further positioned at an angle in a range of 10° to 30° from a vertical plane.

11. The multi-layered sheet mask according to claim 1, wherein the slits of the porous first layer and slits of the porous support structure of the second layer have an inner diameter in a range of 1 μm to 100 μm, and an outer diameter in a range of 100 μm to 150 μm.

12. The multi-layered sheet mask according to claim 1, wherein the slits of the porous first layer and slits of the porous support structure of the second layer have a shape selected from the group consisting of polyhedron, non-polyhedron, and combinations thereof; or
wherein the slits of the porous first layer and slits of the porous support structure of the second layer have a shape selected from the group consisting of cylinder, prism, and combinations thereof; or
wherein the slits of the porous first layer and slits of the porous support structure of the second layer have a hexagonal prism shape.

13. The multi-layered sheet mask according to claim 1, wherein the microchannels of the second layer have a height corresponding to different zones of a face as follows:
a height in the range of 1.5 mm to 1.8 mm corresponding to a first zone consisting of hairline, forehead, temple and combinations thereof of the face;
a height in the range of 1.2 mm to 1.5 mm corresponding to a second zone consisting of nose, cutaneous upper lip, philtum, philtum crest, cutaneous lower lip, chin, and combinations thereof of the face; and
a height in the range of 1.6 mm to 1.9 mm corresponding to a third zone consisting of cheeks, midface, jawline, and combinations thereof of the face, or wherein the microchannels comprise pointed tips which penetrate the second side of the porous first layer, the first side of the porous first layer, an epidermis layer of a skin, and a dermis layer of a skin, when the air pouch is deflated.

14. (canceled)

15. The multi-layered sheet mask according to claim 1, wherein the at least one first active ingredient in each of the at least one pouch of the second layer is the same or different from each other; and
wherein the first active ingredient is selected from the group consisting of anti-pigmentation agent, anti-ageing agent, anti-wrinkle agent, anti-acne agent, moisturizing agent, treatment agent, and mixtures thereof.

16. The multi-layered sheet mask according to claim 1, wherein the third layer is portioned into a number of parts, each part receiving or containing the at least one second active ingredient that is the same or different from each other; and
wherein the second active ingredient is selected from the group consisting of oil, chemical sunscreen, anti-pigmentation agent, anti-ageing agent, anti-wrinkle agent, anti-acne agent, moisturizing agent, treatment agent, and mixtures thereof, or
wherein a circumferential edge comprising the circumferential edges of the porous first layer, the second layer, and the third layer, having a width of at least 5 mm.

17. (canceled)

18. A mould for producing the three-dimensional patterns of the porous first layer of the multi-layered sheet mask according to claim 1, wherein the mould comprises a negative replica of the three-dimensional patterns.

19. The mould according to claim 18, further comprising a plurality of holes in sub-micrometre scale within the negative replica.

20. The mould according to claim 18, wherein the mould is transparent; or wherein the mould is formed by polymerizing a resin over a printed mould comprising a positive replica of the three-dimensional patterns.

21. A method of producing a multi-layered sheet mask, the method comprising the steps of:
a) forming a porous first layer having a first side and a second side, by contacting a mask-shaped biocompatible material with a whole facial mask mould having a plurality of three-dimensional patterns thereon, wherein the three-dimensional patterns comprise a material selected from the group comprising of biocompatible material, exfoliating agent and combinations thereof, and drying for a duration;
b) attaching a second layer to the second side of the porous first layer by bonding the circumferential edges of the first and second layers together; and
c) attaching a third layer to the second layer by bonding the circumferential edges of the second and third layers together;
wherein the multi-layered sheet mask comprises at least
a porous first layer wherein the first side is opposite to the second side and comprises a plurality of three-dimensional patterns extending therefrom;
a second layer attached to the second side of the porous first layer, the second layer comprising at least one pouch for receiving or containing at least one first active ingredient; a porous support structure; an air pouch that is sandwiched between the second side of the porous first layer and the porous support structure; and a plurality of microchannels that extend from the pouch and beyond the first side of the porous first layer when the air pouch is deflated; and
a third layer attached to the second layer for receiving or containing at least one second active ingredient.

22. The method according to claim 21, wherein the three-dimensional patterns of step (a) are formed by filling sections of the whole facial mask mould with the material of step (a) and vacuuming for a duration; and
wherein the sections comprise a mould for producing the three-dimensional patterns of the porous first layer of the multi-layered sheet mask,
wherein the mould comprises a negative replica of the three-dimensional patterns, or
wherein the sections of the whole facial mask mould correspond to facial areas selected from the group consisting of hairline, forehead, temple, nose, cutaneous upper lip, philtum, philtum crest, cutaneous lower lip, chin, cheeks, midface, jawline, and combinations thereof.

23. (canceled)

24. The method according to claim 21, wherein the attaching step (b) further comprises the step of applying fastening strips between the first and second layers; or wherein the attaching step (c) further comprises the step of applying fastening strips between the second and third layers; or wherein the bonding of steps (b) and (c) bonds at least a width of 5 mm of the circumferential edges.

25. (canceled)
